(19) United States
(12) Patent Application Publication
(10) Pub. No.: US 2024/0366241 A1
(43) Pub. Date: Nov. 7, 2024

(54) ASPIRATION CATHETER WITH AUTOMATIC SENSING

(71) Applicant: Boston Scientific Scimed, Inc., Maple Grove, MN (US)

(72) Inventor: Michael P. Schrom, Forest Lake, MN (US)

(73) Assignee: Boston Scientific Scimed, Inc., Maple Grove, MN (US)

(21) Appl. No.: 18/652,935

(22) Filed: May 2, 2024

Related U.S. Application Data
(60) Provisional application No. 63/463,683, filed on May 3, 2023.

(51) Int. Cl. A61B 17/22; A61B 17/00

(52) U.S. Cl. CPC A61B 17/22; A61B 17/00234; A61B 2017/00026; A61B 2017/00238; A61B 2017/00292; A61B 2017/00539; A61B 2017/00778; A61B 2217/005; A61B 2217/007

(57) ABSTRACT

Example medical devices are disclosed herein. An example aspiration catheter includes a catheter body having a proximal end region, a distal end region and a lumen extending therein. The aspiration catheter further includes an impedance sensor disposed along the distal end region of the catheter body, the impedance sensor configured to sense the impedance difference between blood and thrombus.

### CROSS-REFERENCE TO RELATED APPLICATIONS

This application claims the benefit of priority under 35 U.S.C. § 119 of U.S. Provisional Application No. 63/463,683, filed May 3, 2023, the entire disclosure of which is hereby incorporated by reference.

### TECHNICAL FIELD

The disclosure is directed to aspiration systems. More particularly, the disclosure is directed to an aspiration catheter system for improved clot removal. In some instances, the aspiration catheter may include automatic clot sensing and removal.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices. An example aspiration catheter includes a catheter body having a proximal end region, a distal end region and a lumen extending therein. The aspiration catheter further includes an impedance sensor disposed along the distal end region of the catheter body, the impedance sensor configured to sense the impedance difference between blood and thrombus.

Alternatively or additionally to any of the examples above, wherein the impedance sensor includes a first electrode and a second electrode.

Alternatively or additionally to any of the examples above, wherein the first electrode is radially spaced away from the second electrode along the catheter body.

Alternatively or additionally to any of the examples above, wherein the first electrode, the second electrode or both the first electrode and the second electrode are positioned substantially flush with an inner surface of the catheter body.

Alternatively or additionally to any of the examples above, wherein the first electrode, the second electrode or both the first electrode and the second electrode are positioned substantially flush with an outer surface of the catheter body.

Alternatively or additionally to any of the examples above, further comprising a fluid supply tube including at least one proximally projecting jet orifice for expelling at least one proximally oriented fluid jet from the fluid supply tube within the catheter lumen in a generally proximal direction.

Alternatively or additionally to any of the examples above, wherein the fluid supply tube includes at least one distally projecting jet orifice for expelling at least one distally oriented fluid jet from the fluid supply tube within the catheter lumen in a generally distal direction.

Alternatively or additionally to any of the examples above, wherein the at least one distally projecting jet orifice is distal to the at least one proximally projecting jet orifice.

Alternatively or additionally to any of the examples above, wherein the at least one distally projecting jet orifice extends through a sidewall of the fluid supply tube.

Alternatively or additionally to any of the examples above, wherein the catheter body further includes an entrainment inflow orifice positioned along the distal end region of the catheter body.

An example thrombectomy system includes a processor coupled to a pump and a thrombectomy catheter. The thrombectomy catheter includes a catheter body having a proximal end region, a distal end region and a lumen extending therein. The thrombectomy catheter also includes a fluid supply tube extending within the lumen of the catheter body and coupled to the pump. The thrombectomy catheter also includes an impedance sensor disposed along the distal end region of the catheter body. Further, the processor is configured to sense a change in impedance of a first bodily substance adjacent to the impedance sensor.

Alternatively or additionally to any of the examples above, wherein the impedance sensor includes a first electrode and a second electrode.

Alternatively or additionally to any of the examples above, wherein the processor is configured to sense and compare the impedance of the first bodily substance with a second bodily substance.

Alternatively or additionally to any of the examples above, wherein the first bodily substance is blood.

Alternatively or additionally to any of the examples above, wherein the second bodily substance is thrombus.

Alternatively or additionally to any of the examples above, wherein the processor is configured to send a signal to the pump based on the comparison of the impedance of the first bodily substance with the impedance of the second bodily substance.

Alternatively or additionally to any of the examples above, wherein the pump is configured to inject fluid through the fluid supply tube based on the signal received from the processor.

Alternatively or additionally to any of the examples above, wherein the processor is wirelessly coupled to the pump.

An example thrombectomy system includes a processor coupled to a fluid pump and a thrombectomy catheter. The thrombectomy catheter includes a catheter body having a proximal end region, a distal end region and a lumen extending therein. The thrombectomy catheter further includes a fluid supply tube extending within the lumen of the catheter body and coupled to the pump, wherein the fluid supply tube includes at least one proximally projecting jet orifice for expelling at least one proximally oriented fluid jet from the fluid supply tube within the catheter lumen in a generally proximal direction, and wherein the fluid supply tube includes at least one distally projecting jet orifice for expelling at least one distally oriented fluid jet from the fluid supply tube within the catheter lumen in a generally distal direction. The thrombectomy catheter further includes an impedance sensor in communication with the processor, the impedance sensor disposed along the distal end region of the catheter body. Further, the processor is configured to sense the impedance of a bodily substance positioned adjacent to the impedance sensor.

Alternatively or additionally to any of the examples above, the processor is configured to sense the impedance difference between blood and thrombus.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a partially exploded perspective view of a portion of the thrombectomy system of FIG. 1;

FIG. 3 is a partially exploded side view of a portion of the thrombectomy system shown in FIG. 1;

FIG. 4 is a longitudinal cross-sectional view of a distal end region of an illustrative thrombectomy catheter;

FIG. 5A is a schematic illustration of an example thrombectomy catheter system;

FIG. 5B is a perspective view of the distal end region of the thrombectomy catheter shown in FIG. 5A;

FIG. 5C is a perspective view of another example embodiment of the distal end region of the thrombectomy catheter shown in FIG. 5A;

FIG. 6 is a longitudinal cross-sectional view of a distal end region of another illustrative thrombectomy catheter;

FIG. 7 is a longitudinal cross-sectional view of a distal end region of another illustrative thrombectomy catheter;

FIG. 8 is a longitudinal cross-sectional view of a distal end region of another illustrative thrombectomy catheter;

FIG. 9 is a longitudinal cross-sectional view of a distal end region of another illustrative thrombectomy catheter;

FIG. 10 is a longitudinal cross-sectional view of a distal end region of another illustrative thrombectomy catheter;

FIG. 11 is a longitudinal cross-sectional view of a distal end region of another illustrative thrombectomy catheter; and

FIG. 12 is a longitudinal cross-sectional view of a distal end region of another illustrative thrombectomy catheter;

FIG. 13 is a longitudinal cross-sectional view of a distal end region of another illustrative aspiration catheter;

FIG. 14 is a longitudinal cross-sectional view of a distal end region of another illustrative aspiration catheter;

FIG. 15 is a longitudinal cross-sectional view of a distal end region of another illustrative aspiration catheter;

FIG. 16 is a perspective view of the distal end region of the thrombectomy catheter shown in FIG. 15.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. Some thrombectomy catheters may utilize high velocity saline jets in a series to entrain fluid or clot material into and through the shaft of the catheter. Other thrombectomy systems may utilize one or more pressurized saline jets which travel backwards to create a low-pressure zone and a vacuum effect, whereby the vacuum pulls clot material into and through the shaft of the catheter. However, prolonged operation of a thrombectomy system may result in the detrimental removal of excess blood from the veins or arteries of a patient. Accordingly, it may be desirable to design a thrombectomy system which operates (e.g., turns on to remove thrombus, plaque, blood, etc.) only when the distal tip of the system senses the presence of clot material. Thrombectomy systems which operate (e.g., turn on to remove thrombus, plaque, blood, etc.) only when the distal tip of the system senses the presence of clot material are disclosed herein.

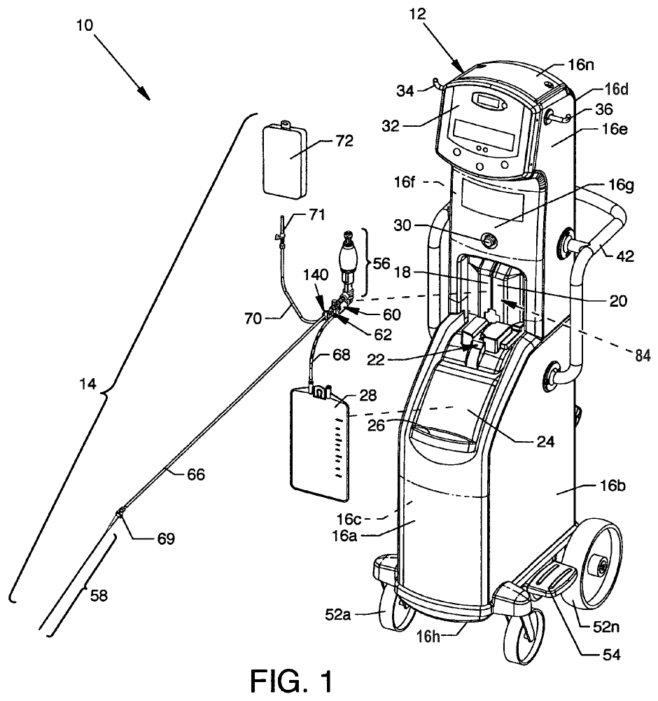

FIG. 1 is a perspective view of an illustrative thrombectomy system 10. The thrombectomy system 10 may include a control console or drive unit 12 and a pump/catheter assembly 14. In some instances, the pump/catheter assembly 14 may be a single use device in which a new pump/catheter assembly 14 may be used with the drive unit 12 for each medical procedure. Shown on the drive unit 12 are a plurality of removable panels 16a-16n about and along the drive unit 12 enclosing the internal structure of the drive unit 12. An illustrative drive unit 12 is described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be automatically opening doors 18 and 20 which open to expose the interior of the drive unit 12 to provide access to a carriage assembly 22. The carriage assembly 22, which may accommodate components of the pump/catheter assembly 14, as discussed further herein, is shown accessible via opening the closed doors 18 and 20. The drive unit 12 may include a catch basin for collecting fluid leakage from the components of the pump/catheter assembly 14. For example, a removable drip tray 24 is shown located on the front of the drive unit 12 extending from below the carriage assembly 22 toward the panel 16a. Other configurations of catch basins are also contemplated. The drip tray 24 and a removable receptacle 26 may collectively support and accommodate an effluent collection bag, such as effluent collection bag 28 of the pump/catheter assembly 14. In other instances, the drive unit 12 may include a different structure, such as a hook for hanging the effluent collection bag 28 from, or a shelf for setting the effluent collection bag 28 on. In instances where the carriage assembly 22 is movable, a carriage assembly activation switch 30 may be provided with the drive unit 12, such as located on panel 16g, to selectively position the carriage assembly 22 inwardly or outwardly. A user interface 32, including memory capabilities, may be provided with the drive unit 12, such as located at the upper region of the drive unit 12 between the upper regions of the upper side panels 16e and 16f. Saline bag hooks 34 and 36 may extend through the panels 16e and 16f to hang saline bags therefrom. The drive unit 12 may include a handle 42 as well as a plurality of wheels 52a-52n and brake pedals 54 for wheel lockage to assist in maneuvering the drive unit 12 by medical personnel.

The pump/catheter assembly 14, which may be a disposable single-use device, is shown unattached from the drive unit 12. The pump/catheter assembly 14 includes a pump 56 and a thrombectomy catheter 58. During use, a portion of the pump/catheter assembly 14 may be secured within a portion of the drive unit 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 attached between the output of the pump 56 and the thrombectomy catheter 58 which may be coaxially arranged inside the effluent return tube 66, a transition fixture 69 between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

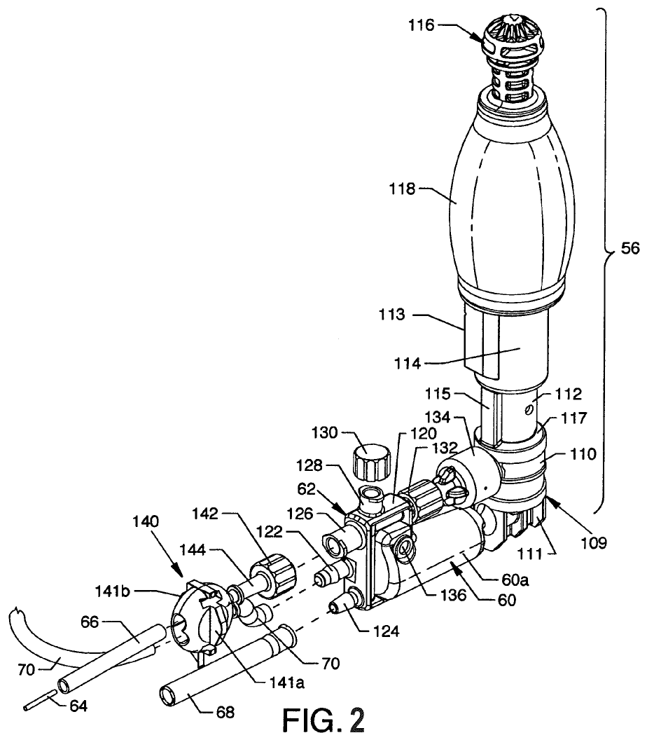

FIG. 2 is a partially exploded perspective view of several components of the pump/catheter assembly 14 generally including the pump 56, the bubble trap 60, the connection manifold assembly 62, and a fixture 140. The pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the 20) carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID tag, or other informational displays to determine operational parameters of the device.

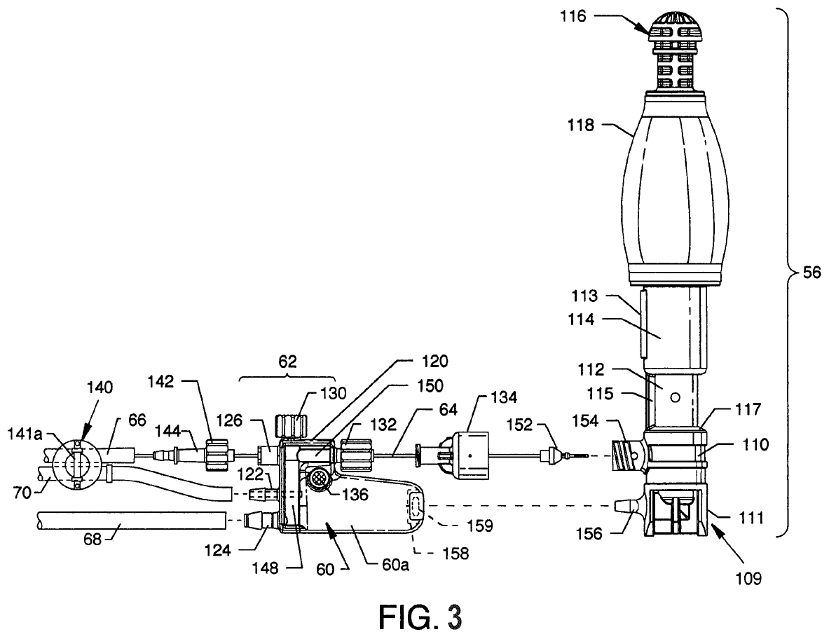

The pump 56 may include a hemispherically-shaped pump piston head 116 having a flexible boot 118 connected to and extending between the top body 114 and the pump piston head 116. In some instances, the geometrically configured lower portion 111 of the base 109 may serve as a mount for one end of the bubble trap 60 (FIG. 3).

The connection manifold assembly 62 may be secured directly to the other end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include mating halves of which one mating half 60a is shown. A hydrophobic filter 136 may be included at the upper forward region of the bubble trap half 60a. Another hydrophobic filter may be included on the second bubble trap half (not explicitly shown) which opposes the hydrophobic filter 136 on the bubble trap half 60a.

The fixture 140, and components associated therewith, assists in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not explicitly shown) provided with the drive unit 12, such as located in the carriage assembly 22 or adjacent thereto.

FIG. 3 is a partially exploded side view of the elements of FIG. 2 illustrating the relationship of the pump 56, the bubble trap 60, the connection manifold assembly 62, and the fixture 140. Also shown is the vertically oriented tubular manifold 148 secured to the bracket 120. The effluent outlet port 124 may be connected to and in fluid communication with the lower interior of the tubular manifold 148. The effluent return port 126 may be connected to and in fluid communication with the upper interior of the tubular manifold 148. Also connecting to the tubular manifold 148 is a horizontally aligned passage port 150 and associated connector 132, each opposing the effluent return port 126. The passage port 150 may accommodate the high-pressure fluid supply tube 64 which extends distally through the lumen (not explicitly shown) of the passage port 150, the connector 132, the upper region of the tubular manifold 148, the effluent return port 126, the connector 142, the connection tube 144, and into and through the effluent return tube 66 to connect to the thrombectomy catheter 58 (FIG. 1). The proximal end of the high-pressure fluid supply tube 64 includes a high-pressure fitting 152 located near the proximal end of the high-pressure fluid supply tube 64 to facilitate connection of the high-pressure fluid supply tube 64 in fluid communication with the interior of the pump 56. The proximal end of the high-pressure fluid supply tube 64, which is the inlet to the high-pressure fluid supply tube 64, may include a plurality of very small holes (not shown) comprising a filter at the proximal end thereof. The connector 134, which may have internal and/or external threads, may be aligned over and about the high-pressure fluid supply tube 64 distal to the high-pressure fitting 152 and threadingly engage a threaded connection port 154 extending horizontally from the upper portion 110 of the base 109 of the pump 56. The connector 134 may be rotated to threadably engage the high-pressure fitting 152 with a corresponding mating threaded structure provided with the pump 56. A connector 132 may be utilized to engage the externally threaded end of the connector 134 to secure the connector 134, and thus the pump 56, to the connection manifold assembly 62 and to provide for fixation of the bubble trap 60 to the pump 56. In addition, direct connection and fluid communication between the pump 56 and the bubble trap 60 may be provided by a horizontally oriented pump fluid inlet port 156 which engages a corresponding receptor port 158 and seal 159 interior to one end of the bubble trap 60. The fluid inlet port 122 located on the bracket 120 may extend behind the tubular manifold 148 to communicate with the interior of the bubble trap 60 for fluid (e.g., saline) debubbling, whereby unpressurized fluid (e.g., saline) is made available for use by the pump 56.

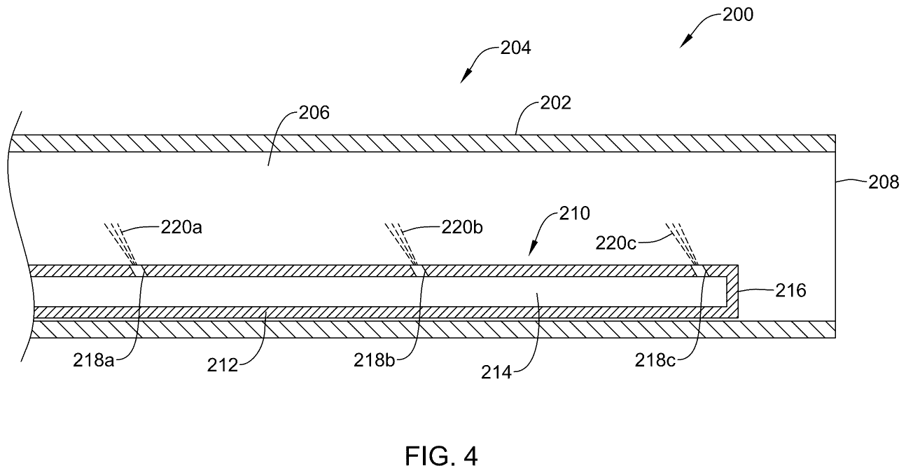

FIG. 4 is a cross-sectional view of a distal end region 204 of an illustrative thrombectomy catheter 200. The thrombectomy catheter 200 may be one illustrative example of the thrombectomy catheter 58 described above. The thrombectomy catheter 200 may include a tubular member or catheter body 202 extending from a proximal end region (not explicitly shown) configured to remain outside the body to a distal end region 204. The catheter body 202 may be one illustrative example of, or be in fluid communication with, the effluent return tube 66 of the thrombectomy catheter 58 described above. A lumen 206 may extend from the proximal end region to the distal end region 204 of the catheter body 202. The catheter body 202 may terminate at a distally facing distal opening 208 at the distal end of the catheter body 202. In some instances, the distal opening 208 may be in a plane that extends generally orthogonal to a longitudinal axis of the catheter body 202. In other instances, the distal opening 208 may be in a plane that extends generally oblique to a longitudinal axis of the catheter body 202. Generally, the distal opening 208 may be an entrainment inflow orifice. While not explicitly shown, the catheter body 202 may include one or more markers (e.g., radiopaque marker bands) disposed along the catheter body 202. Further, while not explicitly shown, in some embodiments, the catheter body 202 may include one or more openings extending through a sidewall thereof, if desired.

The thrombectomy catheter 200 may further include a high-pressure fluid supply tube 210. The high-pressure fluid supply tube 210 may be one illustrative example of, or be in fluid communication with, the high-pressure fluid supply tube 66 of the thrombectomy catheter 58 described above. The high-pressure fluid supply tube 210 may be disposed within and extend through the lumen 206 of the catheter body 202. The high-pressure fluid supply tube 210 may include a supply tube wall 212 defining a lumen or fluid pathway 214 extending therethrough. In at least some instances, the high-pressure fluid supply tube 210 may have a closed distal end 216. Because of this, fluid may be able to pass distally through the fluid pathway 214 but does not exit the distal end. The high-pressure fluid supply tube 210 may extend along a length of the catheter body 202 with the distal end 216 located within the lumen 206 of the catheter body 202 proximal to the distal opening 208 at the distal end of the catheter body 202. A proximal end of the high-pressure fluid supply tube 210 may be in fluid communication with the pump 56 described herein, to provide high-pressure fluid to the fluid pathway 214 of the high-pressure fluid supply tube 210.

A plurality of jet orifices 218a-c (collectively, 218) may be defined along the supply tube wall 212. For example, the supply tube wall 212 may include two, three, four, five, six, or more jet orifices 218. The jet orifices 218 may be spaced along the supply tube wall 212 at any desired intervals. For example, each of the jet orifices 218 may be equidistantly spaced from adjacent jet orifices 218 along the length of the supply tube wall 212. In other instances, the jet orifices 218 may be arranged such that the spacing between adjacent jet orifices 218 near the distal end of the supply tube wall 212 is closer than the spacing between adjacent jet orifices 218 near the proximal end of the supply tube wall 212. For instance, the spacing between the orifices 218 may gradually increase as you move proximally along the length of the shaft, or the spacing may increase in a step-wise configuration. In some instances, some or all of the jet orifices 218 may be axially aligned along the supply tube wall 212. In other instances, one or more of the jet orifices 218 may be circumferentially offset from one another about the supply tube wall 212. A number of patterns are contemplated including a helical pattern, a pattern where no two jet orifices 218 are disposed at the same axial location, a regular pattern including two or more jet orifices 218 disposed at the same axial location, an irregular pattern (where some of the jet orifices 218 may or may not be disposed at the same axial location), etc.

The jet orifices 218 may be formed using a suitable method such as electron discharge machining, etching, cutting (e.g., including laser cutting), or the like. In some instances, one or more of the jet orifices 218 may have a substantially round shape. In other instances, one or more of the jet orifices 218 may have a substantially non-round shape (e.g., oval, polygonal, irregular, etc.). In some instances, the jet orifices 218 may be beveled or otherwise include a beveled surface. It is contemplated that a size and/or a shape of the jet orifices 218 may be varied to vary the velocity of the fluid exiting the jet orifices. For example, decreasing the size of the jet orifices 218 may increase the velocity of the fluid exiting the jet orifices 218. In some embodiments, the size of the jet orifices 218 may be varied based on the pressure capacity of the thrombectomy system, the number of jet orifices, the dimensions of the high-pressure fluid supply tube 210 (e.g., length, wall thickness, inner diameter, etc.), and/or combinations thereof. In some examples, the jet orifices 218 may have a cross-sectional dimension in the range of about 0.0018 inches (45.72 micrometers) to about 0.0022 inches (55.88 micrometers). However, the jet orifices 218 can have a cross-sectional dimension of less than 0.0018 inches (45.72 micrometers) or greater than 0.0022 inches (55.88 micrometers), as desired.

Infusion of motive fluid through the lumen 214 of the supply tube wall 212 may result in fluid being jetted through the jet orifices 218 and the generation of a proximally directed aspiration force. At least some of the jet orifices 218a-c may be angled in a proximal direction or otherwise designed to infuse fluid (e.g., a motive fluid, a liquid, a gas or air, steam, a fluid with particles disposed therein, or the like) through the jet orifices 218a-c and into the lumen 206 of the catheter body 202 in a generally proximal direction as depicted by lines 220a-c representing motive jetted fluid projecting generally proximally from the jet orifices 218a-c. For example, each of the jet orifices 218a-c may be arranged at an acute angle to the longitudinal axis of the supply tube wall 212 such that the jet orifices 218a-c angle in a proximal direction. It is contemplated that an angle of the jet orifices 218 and thus the motive jetted fluid 220 may be varied to adjust the velocity of the fluid exiting the jet orifices 218.

The performance of the thrombectomy catheter 200 and the high-pressure fluid supply tube 210 may be directly related to the velocity of the motive jetted fluid 220 exiting the jet orifices 218 and the shear-induced turbulent flux created by the jetted motive fluid 220. For example, the more powerful the jetted motive fluid 220, the higher the aspiration rates may be. It is further contemplated that the performance of the jet-powered aspiration catheter 200 may be directly related to the speed at which the clot can be entrained into the catheter 200, macerated, and removed from the body. Any clogging that occurs within the catheter body 202 may reduce or completely stop the removal of the clot. It is contemplated that the properties (size, shape, angle, number, spacing, etc.) of the jet orifices 218 may be varied to obtain a fluid velocity that creates an optimum de-clogging effect without hindering the proximal flow of a clot within the lumen 206 of the catheter body 202 or the clot evacuation rate.

In some instances, the jet orifices 218 may be oriented at an angle relative to the longitudinal axis of the supply tube wall 212. For example, the proximally projecting jet orifices 218a-c may be oriented at an oblique (e.g., acute) angle relative to the longitudinal axis of the supply tube wall 212 and/or oriented at an angle greater than zero degrees and less than ninety degrees relative to the longitudinal axis of the supply tube wall 212. In other instances, the jet orifices 218 may be oriented perpendicular to the longitudinal axis of the supply tube wall 212 (e.g., at an angle of about 90 degrees relative to the longitudinal axis of the supply tube wall 212). The angle may or may not be the same for all the jet orifices 218.

In at least some instances, the jet orifices 218 may be understood as being arranged in series. In other words, the jet orifices 218 may be arranged such that adjacent jet orifices 218 are spaced longitudinally apart at various locations along the longitudinal axis of the supply tube wall 212. For example, the jet orifices 218 may be uniformly or non-uniformly spaced along of a length of the supply tube wall 212. This may position the jet orifices 218 at axially spaced apart locations within the catheter body 202 and along the length thereof. For example, the jet orifices 218 may be spaced along an entire length of the supply tube wall 212 and correspondingly along an entire length of the catheter body 202, or portions thereof, as desired. In some examples, the jet orifices 218 may be spaced at intervals in the range of every 5 inches (12.7 centimeters (cm)) to every 15 inches (38.1 cm), or in the range of every 6 inches (15.2 cm) to every 12 inches (30.5 cm) along a length of the supply tube wall 212. In other instances, the spacing between the jet orifices 218 may be less than every 5 inches (12.7 cm) or greater than every 15 inches (38.1 cm). Accordingly, motive fluid leaves via the jet orifices 218 forming a jetted motive fluid 220a-c (collectively, 220). In some instances, the jetted motive fluid 220 may reach speeds of 17,150 centimeters/second or greater (e.g., half the speed of sound, or greater). This jetted motive fluid 220 enters an entrainment material where the shear layer between the two causes turbulence, mixing, and transfer of momentum. Entrainment material may enter the distal opening 208 and then may be urged proximally by momentum transfer. As the mixture of jetted motive fluid 220 and entrainment material migrates proximally, the material may sequentially approach a number of jet orifices 218. Upon interaction with the jetted motive fluid 220 from each individual jet orifice 218, the momentum in the entrainment material mixture may increase, and the thrombogenic material may more readily flow proximally through the catheter body 202 for removal. The increase in momentum may allow for the catheter body 202 to be used without a second or outflow orifice (e.g., positioned proximally of the distal opening 208). Alternatively, some of the entrapped thrombogenic material may exit the catheter body 202 through a second orifice (not shown), e.g., in a sidewall of the catheter body 202, positioned proximal to the distal opening 208, recirculate to the distal opening 208 (e.g., one or more times), and then move proximally through the lumen 206 of the catheter body 202.

As discussed herein, in some instances it may be desirable to design the thrombectomy systems and components thereof disclosed herein (e.g., system 10, thrombectomy catheter 200, etc.) to include one or more features which permit the thrombectomy system 10 to sense (e.g., detect) the presence of a clot, plaque, thrombus, etc. and automatically start the thrombectomy system 10 to remove the clot, plaque, thrombus, etc. and automatically stop the thrombectomy system 10 upon removal of the clot, plaque, thrombus, etc. to limit and/or prevent excess blood from being removed from the patient.

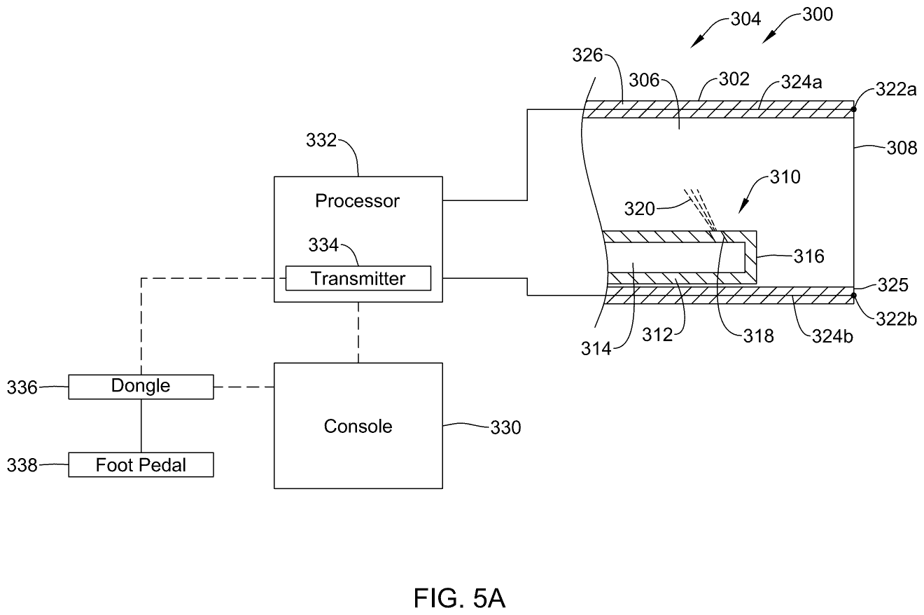

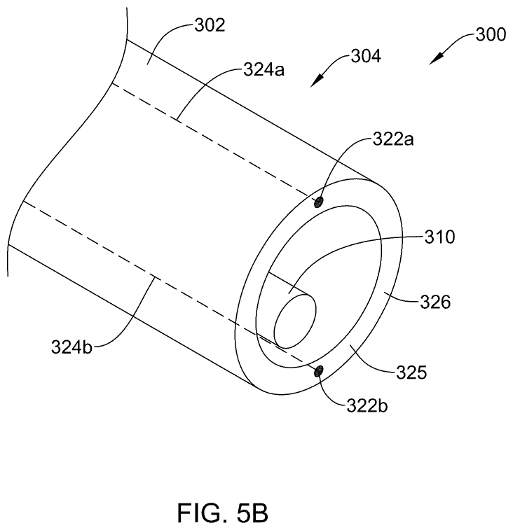

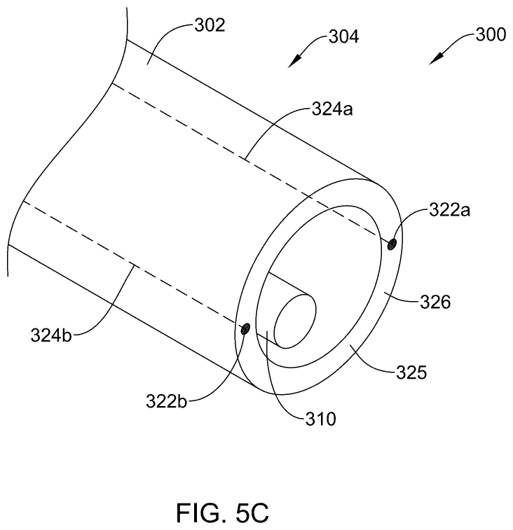

FIG. 5A illustrates a cross-sectional view of the distal end region of an example thrombectomy catheter 300. The thrombectomy catheter 300 may be similar in form and function to any of thrombectomy catheters disclosed herein (e.g., the catheter 58, catheter 200, etc.). For example, the thrombectomy catheter 300 may include a tubular member or catheter body 302 extending from a proximal end region (not explicitly shown) configured to remain outside the body to a distal end region 304. The catheter body 302 may be one illustrative example of, or be in fluid communication with, the effluent return tube 66 of the thrombectomy catheter 58 described above. A lumen 306 may extend from the proximal end region to the distal end region 304 of the catheter body 302. The catheter body 302 may terminate at a distally facing distal opening 308 at the distal end of the catheter body 302. In some instances, the distal opening 308 may be in a plane that extends generally orthogonal to a longitudinal axis of the catheter body 302. In other instances, the distal opening 308 may be in a plane that extends generally oblique to a longitudinal axis of the catheter body 302.

The thrombectomy catheter 300 may further include a high-pressure fluid supply tube 310. The high-pressure fluid supply tube 310 may be one illustrative example of, or be in fluid communication with, the high-pressure fluid supply tube 66 of the thrombectomy catheter 58 described above. The high-pressure fluid supply tube 310 may be disposed within and extend through the lumen 306 of the catheter body 302. The high-pressure fluid supply tube 310 may include a supply tube wall 312 defining a lumen or fluid pathway 314 extending therethrough. In at least some instances, the high-pressure fluid supply tube 310 may have a closed distal end 316. Because of this, fluid may be able to pass distally through the fluid pathway 314 but does not exit the distal end. The high-pressure fluid supply tube 310 may extend along a length of the catheter body 302 with the distal end 316 located within the lumen 306 of the catheter body 302 proximal to the distal opening 308 at the distal end of the catheter body 302. A proximal end of the high-pressure fluid supply tube 310 may be in fluid communication with the pump 56 described herein, to provide high-pressure fluid to the fluid pathway 314 of the high-pressure fluid supply tube 310.

FIG. 5A illustrates at the thrombectomy catheter 300 may include one or more jet orifices 318 which may be defined along the supply tube wall 312. While only one jet orifice is depicted in FIG. 5A, it can be appreciated that the supply tube wall 312 may include one, two, three, four, five, six, or more jet orifices 318 (similar in form and function to the jet orifices 218a-c disclosed above with respect to the thrombectomy catheter 200). Like that described herein with respective the catheter 200, infusion of motive fluid through the lumen 314 of the supply tube wall 312 may result in fluid being jetted through the jet orifices (e.g., the one or more jet orifices 318) and the generation of a proximally directed aspiration force through the catheter body 312. Further, entrainment material may enter the distal opening 308 and then may be urged proximally by momentum transfer. As the mixture of jetted motive fluid 320 and entrainment material moves proximally, the material may sequentially approach a number of jet orifices (e.g., the one or more jet orifices 318) positioned along the supply tube wall 312. Upon interaction with the jetted motive fluid 320 from each individual jet orifice 318, the momentum in the entrainment material mixture may increase, and the thrombogenic material may more readily flow proximally through the catheter body 302 for removal.

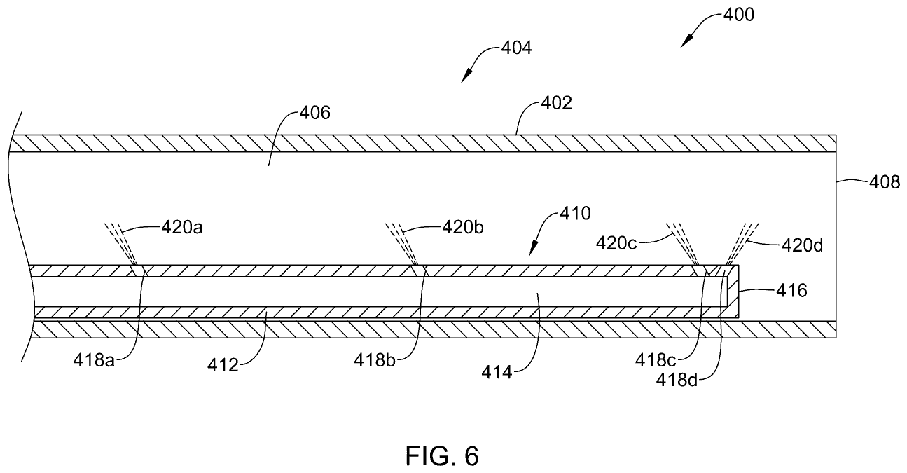

FIG. 5A further illustrates that the thrombectomy catheter 300 may include a first 20) electrode 322a positioned within the wall 326 of the catheter body 320 and a second electrode 322b positioned within the wall 326 of the catheter body 320. It can be appreciated that the first electrode 322a may be radially, axially and/or circumferentially spaced (e.g., offset, etc.) from the second electrode 322b at any position (e.g., longitudinal position) along the catheter body 302. FIG. 5A further illustrates that the first electrode 322a and the second electrode 322b maybe positioned along a distal facing surface 325 of the catheter body 302 (the position of the first electrode 322a and the second electrode 322b along the distal facing surface 325 is further illustrated in FIG. 6). It can be appreciated that while FIG. 5A illustrates that the first electrode 322a and/or the second electrode 322b may be positioned along the distal facing surface 325 of the catheter body 302, the first electrode 322a and/or the second electrode 322b may be positioned axially along any portion of the distal end region 304 of the catheter body 302. For example, the first electrode 322a and/or the second electrode 322b may be positioned at any location proximally of the distal end of the catheter body 302 (e.g., the first electrode 322a and/or the second electrode 322b may be positioned within the wall 326 of the proximal body 302 at any position proximal of the distal facing surface 325 of the catheter body 302).

Further, FIG. 5A illustrates that the first electrode 322a may be attached to a first wire 324a. The first wire 324a may be positioned within the wall 326 of the catheter body 302. Additionally, FIG. 5A illustrates that the second electrode 322b may be attached to a second wire 324b. The second wire 324b may also be positioned within the wall 326 of the catheter body 302.

While FIG. 5A illustrates that the first electrode 322a and the second electrode 322b positioned within the wall 326 of the catheter body 302, it can be appreciated that the first electrode 322a, the second electrode 322b or both the first electrode 322a and the second electrode 322b may be positioned substantially flush with an inner surface of the catheter body 302. Further, it can be appreciated that the first electrode 322a, the second electrode 322b or both the first electrode 322a and the second electrode 322b may be positioned substantially flush with an outer surface of the catheter body 302. Further, it can be appreciated that the first wire 324a, the second wire 324b or both the first wire 324a and the second wire 324b may be positioned substantially flush with an inner surface of the catheter body 302. Further, it can be appreciated that the first wire 324a, the second wire 324b or both the first wire 324a and the second wire 324b may be positioned substantially flush with an outer surface of the catheter body 302.

FIG. 5A further illustrates that the first wire 324a and the second wire 324b may extend way from the first electrode 322a, 322b, respectively, through the wall 326 of the catheter body and eventually be coupled (e.g., be attached) to a processor 332. FIG. 5A illustrates that the processor 332 may be coupled to a console 330. In some examples, the console 330 may be defined as a simple switch (or other similar component) designed to open/close an electrical circuit an electrical circuit which starts/stops the pump 56. It can be appreciated that, in some examples, the processor 332 may be a distinct component spaced away from the console 330. The processor 332 may be configured to communicate wirelessly (e.g., via Bluetooth communication, etc.) with the console 330. In other examples, the processor 332 may communicate with the console 330 via a hardwire connection. It can be further appreciated that, in other examples, the processor 332 may be integrated into the console 330. In other words, the processor 332 may be located within the control console or drive unit 12 shown in FIG. 1.

FIG. 5A further illustrates that the processor 332 may include a signal transmitter 334. Additionally, the signal transmitter 334 may be configured to transport a signal (or create a pathway for a signal) to travel from the processor 332 to the console 330 or a component coupled to the console 330. For example, FIG. 5A illustrates that the transmitter 334 may be configured to send a signal to a dongle 336 which is coupled to the console 330. As will be described in greater detail herein, the signal may trigger the dongle 336 to open or close an electrical circuit which starts/stops the pump 56. FIG. 5A further illustrates that a foot pedal 338 may also be coupled to the dongle 338, whereby the foot pedal 338 is configured to send a signal to the dongle 336. The signal sent from the foot pedal 338 to the dongle 336 may cause the dongle 336 to open or close an electrical circuit which starts/stops the pump 56 within the console 330.

Additionally, the first electrode 322a and the second electrode 322b may operate in a bipolar configuration to sense (e.g., measure, detect, etc.) a change in a parameter (e.g., impedance, pressure, force, etc.) of a bodily substance (e.g., blood, tissue, plaque, thrombus, clot, etc.) positioned between the first electrode 322a and the second electrode 322b. For example, it can be appreciated that thrombectomy system 10 may be configured 20) to send an electrical signal from the processor 332 to the first electrode 324a via the first wire 324a, whereby the electrical signal passes from the first electrode 324a, through the bodily substance present at the distal end region of the catheter body 302 (e.g., through the bodily substance positioned between the first electrode 322a and the second electrode 322b), whereby the electrical signal is then received by the second electrode 322b. It can further be appreciated that the electrical signal may then be passed from the second electrode 322b back to the processor 332 via the second wire 324b. Both the first electrode 322a and the second electrode 322b electrodes contribute to the measured impedance because the electrical current passes from one electrode to the other through the small volume of bodily substance between them. The processor 332 may be configured to compare the impedance sensed by the first electrode 322a and the second electrode 322b, whereby the processor 332 may sense a relative change (e.g., the delta) in the impedance sensed by the first electrode 322a and the second electrode 322b of a bodily substance positioned adjacent to the first electrode 322a and the second electrode 322b.

Additionally, the processor 332 may be programmed to compare the signal sent from the processor 332 to first electrode 322a to the signal received from the second electrode 322b. The resultant value may provide a measurement of the impedance (e.g., opposition to electrical flow) of the bodily substance positioned between the first electrode 322a and the second electrode 322b. Further, the processor 332 may be configured to compare a measured impedance to an approximate, preset (e.g., preprogrammed) ranges for the impedance of blood versus the impedance of a clot (e.g., plaque, thrombus, etc.). The preset (e.g., preprogrammed) ranges for the impedance of blood versus the impedance of a clot (e.g., plaque, thrombus, etc.) may be stored in the memory or processing componentry 332 of the system 10. In other examples, the ranges for the impedance of blood versus the impedance of a clot (e.g., plaque, thrombus, etc.) may be input into the system 10 by a clinician via a touchpad on the console 330. In other words, the processor 332 may be configured to compare the impedance sensed by the first electrode 322a and the second electrode 322b to a preprogrammed value, whereby the processor 332 may sense the relative change (e.g., the delta) between the impedance sensed by the first electrode 322a and the second electrode 322b to preprogrammed values of a bodily substance positioned adjacent to the first electrode 322a and the second electrode 322b.

It can be further appreciated that the system 10 may be configured such that when the measured impedance change is within a range which indicates the tip of the catheter 300 is adjacent to a clot (e.g., plaque, thrombus, etc.), the transmitter 334 may send a signal (e.g., a wireless signal) to the dongle 336, whereby the dongle 336 may close an electrical circuit which than starts the pump 56. As discussed herein, starting the pump 56 may cause infusion of motive fluid through the lumen 314 of the supply tube wall 312, resulting in fluid being jetted through the jet orifices (e.g., the one or more jet orifices 318) and the generation of a proximally directed aspiration force through the catheter body 312 to remove the clot (e.g., plaque, thrombus, etc.).

In other examples, the catheter 300 may include one or more blood-sensing electrodes and/or sensors positioned along the catheter 300 whereby the blood-sensing electrodes or sensors may be adjacent to blood when the distal tip of the catheter 300 is adjacent to a clot (e.g., plaque, thrombus, etc.). The blood-sensing electrodes and/or sensors may be similar in form and function to the first electrode 322a and/or the second electrode 322b. For example, like the first electrode 322a and the second electrode 322b, the blood-sensing electrodes may be able to measure the impedance of blood when the distal tip of the catheter 300 is adjacent to a clot (e.g., plaque, thrombus, etc.). Further, the system 10 may be configured to compare the impedance of material adjacent to the distal tip of the catheter 300 to the impedance of blood measured by the blood-sensing electrodes. Accordingly, the processor 332 may be configured to measure and compute the ratio of the impedance of material adjacent to the distal tip of the catheter 300 compared to the impedance of blood measured by the blood-sensing electrodes. Further, this resultant ratio value may indicate that the tip of the catheter 300 is adjacent to a clot (e.g., plaque, thrombus, etc.). For example, the processor 332 may be configured to compare this calculated ratio to approximate, preset (e.g., preprogrammed) ranges for the ratio of the impedance of material adjacent to the distal tip of the catheter 300 compared to the impedance of blood measured by the blood-sensing electrodes. The preset (e.g., preprogrammed) ranges for the calculated ratio may be stored in the memory or processing componentry 332 of the system 10. In other examples, the calculated ration may be input into the system 10 by a clinician via a touchpad on the console 330.

It can be further appreciated that the system 10 may be configured such that when the calculated ratio is within a range which indicates the tip of the catheter 300 is adjacent to a clot (e.g., plaque, thrombus, etc.), the transmitter 334 may send a signal (e.g., a wireless signal) to the dongle 336, whereby the dongle 336 may close an electrical circuit which than starts the pump 56. As discussed herein, starting the pump 56 may cause infusion of motive fluid through the lumen 314 of the supply tube wall 312, resulting in fluid being jetted through the jet orifices (e.g., the one or more jet orifices 318) and the generation of a proximally directed aspiration force through the catheter body 312 to remove the clot (e.g., plaque, thrombus, etc.).

It can be appreciated that after the thrombectomy catheter 300 has removed the clot, plaque, thrombus, etc., blood may fill the distal tip of the catheter 300 between the first electrode 322a and the second electrode 322a may be blood, and therefore, the impedance measurement sensed by processor 332 may change. Accordingly, when the measured impedance is within a range which indicates the tip of the catheter 300 is adjacent to a blood (which has a different impedance than clot, plaque, thrombus), the transmitter 334 may send a signal (e.g., a wireless signal) to the dongle 336, whereby the dongle 336 may open an electrical circuit which than stops the pump 56. As discussed herein, stopping the pump 56 may stop the infusion of motive fluid through the lumen 314 of the supply tube wall 312, preventing the continued removal of blood from the patient.

Additionally, it can be appreciated that, in some examples, the signal transmitter 334 may be configured to transport a signal (or create a pathway for a signal) to travel from the processor 332 directly to the console 330 (bypassing the dongle 336, for example). For example, FIG. 5A illustrates that the transmitter 334 may be configured to send a signal directly to a receiver positioned in the console 330. As will be described in greater detail herein, the signal may trigger the receiver (or another component within the console 330 that receives the signal) to start/stop the pump 56 within the console 330. Further, in some examples, the drive unit 12 of the thrombectomy system 10 may plug directly into the console 330, whereby the drive unit 12 may transmit a signal which triggers a receiver (or other component within the console 330 that receives the signal) to start/stop the pump 56 within the console 330. In yet other examples, the console 330 may include a Wi-Fi adaptor, whereby the Wi-Fi adaptor is configured to receive a signal from another component of the thrombectomy system 10 (e.g., the drive unit 12, the processor 332, the transmitter 334, etc.), and whereby the signal received by the Wi-Fi adaptor may trigger the starting or stopping of the pump 56.

It can be further appreciated that when the system 10 may be defined as being configured to “automatically” sense the presence of clot, plaque, thrombus or the like when the system 10 is configured such that the processor continuously senses the impedance of the bodily material positioned adjacent the distal tip of the catheter 300 (e.g., senses the impedance of bodily material between the first electrode 322a and the second electrode 322b), and subsequently causes the transmitter to send signals to the console 330 and/or dongle 336 to turn the pump 56 on/off. In some instances, the console 330 may include a switch, button, actuator, etc. which permits a clinician to turn the “automatic” impedance sensing operation of the system 10 on or off. For example, the console 336 may include an on/off switch which must be turned on by the clinician in order for the system 10 to operate in the “automatic impedance sensing” configuration, whereby by the pump 56 is automatically turned on and off depending on the impedance measurements sensed by the processor 332.

The processor 332 may be configured to continually measure the impedance of bodily material positioned between the first electrode 322a and the second electrode 322b. Accordingly, the processor 332 may be configured to receive and process impedance signals such that it may start and stop the pump 56 with a single pump stroke.

As discussed herein, the system 10 may be designed to include a foot pedal 338 coupled to the dongle 336. It can be appreciated that the foot pedal 338 may permit a clinician to activate the pump 56, and hence, the flow fluid through the lumen 314. In some instances, the foot pedal 338 may be utilized to override a signal coming from the processor 332 (via the transmitter 334). For example, in some instances when the clinician is operating the system 10 in the “automatic” operating configuration as described herein, the clinician may want to activate the pump 56 despite the processor 332 not having sensed an impedance which would automatically active that pump 56. In other words, depressing the foot pedal 338 may permit the clinician to manually activate the pump 56 even though the system 10 is in the “automatic” operating configuration as described herein.

FIG. 5B is a perspective view of the distal end region 304 of the example thrombectomy catheter 300 shown in FIG. 5A. As discussed herein, the thrombectomy catheter 300 may include a tubular member or catheter body 302 extending from a proximal end region (not explicitly shown) configured to remain outside the body to a distal end region 304. FIG. 5B further illustrates the high-pressure fluid supply tube 310 discussed herein.

As discussed herein, FIG. 5B further illustrates the first electrode 322a positioned on the distal facing surface 325 of the wall 326 of the catheter body 302 and a second electrode 322b positioned on the distal facing surface 325 of the wall 326 of the catheter body 302. It can be appreciated that the first electrode 322a may be radially, axially and/or circumferentially spaced (e.g., offset, etc.) from the second electrode 322b at any position (e.g., longitudinal position) along the distal facing surface 325 of the catheter body 302.

Using the analogy of a 12-hour clock face, FIG. 5B illustrates that the first electrode 322a and/or the second electrode 322b may be positioned along the distal facing surface 325 at approximately a 12 o'clock and 6 o'clock position. However, this is not intended to be limiting. Rather, for any of the example aspiration and/or thrombectomy systems described herein, the first electrode 322a and/or the second electrode 322b may be positioned at any location along the distal face 325 or within the wall 326 of the catheter body 326. For example, FIG. 5C illustrates that the first electrode 322a and/or the second electrode 322b may be positioned along the distal facing surface 325 or within the wall 326 of the catheter body 326 at approximately a 9 o'clock and 3 o'clock position, respectively.

FIG. 6 is a cross-sectional view of a distal end region 404 of another illustrative thrombectomy catheter 400. The thrombectomy catheter 400 may be one illustrative example of the thrombectomy catheter 58 described above. The thrombectomy catheter 400 may include a tubular member or catheter body 402 extending from a proximal end region (not explicitly shown) configured to remain outside the body to a distal end region 404. The catheter body 402 may be one illustrative example of, or be in fluid communication with, the effluent return tube 66 of the thrombectomy catheter 58 described above. A lumen 406 may extend from the proximal end region to the distal end region 404 of the catheter body 402. The catheter body 402 may terminate at a distally facing distal opening 408 at the distal end of the catheter body 402. In some instances, the distal opening 408 may be in a plane that extends generally orthogonal to a longitudinal axis of the catheter body 402. In other instances, the distal opening 408 may be in a plane that extends generally oblique to a longitudinal axis of the catheter body 402. Generally, the distal opening 408 may be an entrainment inflow orifice. While not explicitly shown, the catheter body 402 may include one or more markers (e.g., radiopaque marker bands) disposed along the catheter body 402. Further, while not explicitly shown, in some embodiments, the catheter body 402 may include one or more openings extending through a sidewall thereof, if desired.

The thrombectomy catheter 400 may further include a high-pressure fluid supply tube 410. The high-pressure fluid supply tube 410 may be one illustrative example of, or be in fluid communication with, the high-pressure fluid supply tube 66 of the thrombectomy catheter 58 described above. The high-pressure fluid supply tube 410 may be disposed within and extend through the lumen 406 of the catheter body 402. The high-pressure fluid supply tube 410 may include a supply tube wall 412 defining a lumen or fluid pathway 414 extending therethrough. In at least some instances, the high-pressure fluid supply tube 410 may have a closed distal end 416. Because of this, fluid may be able to pass distally through the fluid pathway 414 but does not exit the distal end. The high-pressure fluid supply tube 410 may extend along a length of the catheter body 402 with the distal end 416 located within the lumen 406 of the catheter body 402 proximal to the distal opening 408 at the distal end of the catheter body 402. A proximal end of the high-pressure fluid supply tube 410 may be in fluid communication with the pump 56 described herein, to provide high-pressure fluid to the fluid pathway 414 of the high-pressure fluid supply tube 410.

A plurality of jet orifices 418a-d (collectively, 418) may be defined along the supply tube wall 412. For example, the supply tube wall 412 may include two, three, four, five, six, or more jet orifices 418. The jet orifices 418 may be spaced along the supply tube wall 412 at any desired intervals. For example, each of the jet orifices 418 may be equidistantly spaced from adjacent jet orifices 418 along the length of the supply tube wall 412. In other instances, the jet orifices 418 may be arranged such that the spacing between adjacent jet orifices 418 near the distal end of the supply tube wall 412 is closer than the spacing between adjacent jet orifices 418 near the proximal end of the supply tube wall 412. For instance, the spacing between the orifices 418 may gradually increase as you move proximally along the length of the shaft, or the spacing may increase in a step-wise configuration. In some instances, some or all of the jet orifices 418 may be axially aligned along the supply tube wall 412. In other instances, one or more of the jet orifices 418 may be circumferentially offset from one another about the supply tube wall 412. A number of patterns are contemplated including a helical pattern, a pattern where no two jet orifices 418 are disposed at the same axial location, a regular pattern including two or more jet orifices 418 disposed at the same axial location, an irregular pattern (where some of the jet orifices 418 may or may not be disposed at the same axial location), etc.

The jet orifices 418 may be formed using a suitable method such as electron discharge machining, etching, cutting (e.g., including laser cutting), or the like. In some instances, one or more of the jet orifices 418 may have a substantially round shape. In other instances, one or more of the jet orifices 418 may have a substantially non-round shape (e.g., oval, polygonal, irregular, etc.). In some instances, the jet orifices 418 may be beveled or otherwise include a beveled surface. It is contemplated that a size and/or a shape of the jet orifices 418 may be varied to vary the velocity of the fluid exiting the jet orifices. For example, decreasing the size of the jet orifices 418 may increase the velocity of the fluid exiting the jet orifices 418. In some embodiments, the size of the jet orifices 418 may be varied based on the pressure capacity of the thrombectomy system, the number of jet orifices, the dimensions of the high-pressure fluid supply tube 410 (e.g., length, wall thickness, inner diameter, etc.), and/or combinations thereof. In some examples, the jet orifices 418 may have a cross-sectional dimension in the range of about 0.0018 inches (45.72 micrometers) to about 0.0022 inches (55.88 micrometers). However, the jet orifices 418 can have a cross-sectional dimension of less than 0.0018 inches (45.72 micrometers) or greater than 0.0022 inches (55.88 micrometers), as desired.

Infusion of motive fluid through the lumen 414 of the supply tube wall 412 may result in fluid being jetted through the jet orifices 418 and the generation of a proximally directed aspiration force. At least some of the jet orifices 418a-c may be angled in a proximal direction or otherwise designed to infuse fluid (e.g., a motive fluid, a liquid, a gas or air, steam, a fluid with particles disposed therein, or the like) through the jet orifices 418a-c and into the lumen 406 of the catheter body 402 in a generally proximal direction as depicted by lines 420a-c representing motive jetted fluid projecting generally proximally from the jet orifices 418a-c. For example, each of the jet orifices 418a-c may be arranged at an acute angle to the longitudinal axis of the supply tube wall 412 such that the jet orifices 418a-c angle in a proximal direction. In some embodiments, one or more of the jet orifices 418d may be designed to infuse fluid (e.g., a motive fluid, a liquid, a gas or air, steam, a fluid with particles disposed therein, or the like) through the jet orifice(s) 418d and into the lumen 406 of the catheter body 402 in a generally distal direction as depicted by lines 420d representing motive jetted fluid projecting generally distally from the jet orifice 418d. For example, the jet orifice 418d may be arranged at an oblique angle to the longitudinal axis of the supply tube wall 412 such that the jet orifice 418d angles in a distal direction. It is contemplated that an angle of the jet orifices 418 and thus the motive jetted fluid 420 may be varied to adjust the velocity of the fluid exiting the jet orifices 418. As further described herein, the supply tube wall 412 may include one or more, or a plurality of proximally oriented or directed jet orifices 418a, 418b, 418c (i.e., jet orifices configured to direct fluid infused through the lumen 414 of the supply tube wall 412 in a proximal direction) and the supply tube wall 412 may include one or more, or a plurality of distally oriented or directed jet orifices 418d (i.e., jet orifices configured to direct fluid infused through the lumen 414 of the supply tube wall 412 in a distal direction). In some examples, the distally projecting jet orifice 418d may be axially aligned with one or more of the proximally projecting jet orifices 418a-c. In other examples, the distally projecting jet orifice 418d may be circumferentially offset from one or more of the proximally projecting jet orifices 418a-c. For example, the distally projecting jet orifice 418d may be circumferentially offset from one or more of the proximally projecting jet orifices 418a-c by in the range of about 10° to about 350° or about 45° to about 135°.

The distally projecting jet orifice 418d may be the distalmost jet orifice, with the proximally projecting jet orifices 418a-c positioned proximal of the distally projecting jet orifice 418d. However, this is not required. In some embodiments, the distally projecting jet orifice 418d may be positioned proximal to at least one proximally projecting jet orifice 418a-c. While the supply tube wall 412 is illustrated as including only a single distally projecting jet orifice 418d, the supply tube wall 412 may include more than one distally projecting jet orifice, as desired. When more than one distally projecting jet orifice 418d is provided, the distally projecting jet orifices may be positioned at differing axial and/or circumferential locations from one another or similar axial and/or circumferential locations 20) as one another, as desired. The distally projecting jet orifice(s) 418d may break up particles as they are drawn into the lumen 406 of the catheter body 402 while the proximally projecting jet orifices 418a-c may move particles proximally along the catheter body 402.

The performance of the thrombectomy catheter 400 and the high-pressure fluid supply tube 410 may be directly related to the velocity of the motive jetted fluid 420 exiting the jet orifices 418 and the shear-induced turbulent flux created by the jetted motive fluid 420. For example, the more powerful the jetted motive fluid 420, the higher the aspiration rates may be. It is further contemplated that the performance of the jet-powered aspiration catheter 400 may be directly related to the speed at which the clot can be entrained into the catheter 400, macerated, and removed from the body. Any clogging that occurs within the catheter body 402 may reduce or completely stop the removal of the clot. The addition of the distally projecting jet orifice 418d may macerate any clot that enters the distal opening 408 of the catheter body 402 thus helping prevent clogging. For example, at the point of impingement of the distally oriented motive jetted fluid 420d the motive jetted fluid 420d may deflect distally creating flow out the tip of the distal opening 408 of the catheter body 402, effectively macerating any clot that enters the tip of the device and eliminating or reducing risk of the distal opening 408 of the catheter body 402 becoming blocked or clogged. It is contemplated that the properties (size, shape, angle, number, spacing, etc.) of the jet orifices 418 may be varied to obtain a fluid velocity that creates an optimum de-clogging effect without hindering the proximal flow of a clot within the lumen 406 of the catheter body 402 or the clot evacuation rate.

The distally projecting jet orifice 418d may be proximally spaced a distance from the distal opening 408 of the catheter body 402. It is contemplated that the longitudinal location of the distally projecting jet orifice 418d on the supply tube wall 412 and relative to the distal opening 408 of the catheter body 402 may be varied based on a size of the aperture of the distally projecting jet orifice 418d, the velocity of the fluid within the lumen 414 of the supply tube wall 412, the angle of the distally projecting jet orifice 418d, or combinations thereof, etc. to ensure the distally oriented motive jetted fluid 420d impinges the inner surface of the catheter body 402. In one illustrative example, the distally projecting jet orifice 418d may be positioned such that the distally oriented motive jetted fluid 420d impinges an inner surface of the catheter body 402 such that the distally oriented motive jetted fluid 420d does not damage the vessel. For example, the distally projecting jet orifice 418d may be positioned such that the distally oriented motive jetted fluid 420d impinges an inner surface of the catheter body 402 in the range of about 0.070 inches (1.778 millimeters) to about 0.090 inches (2.286 millimeters) proximal to the distal end of the catheter body 402. This is just one example. The impingement location of the motive jetted fluid 420d of the distally projecting jet orifice 418d may be less than 0.070 inches (1.778 millimeters) or more than 0.090 inches (2.286 millimeters) proximal to the distal end of the catheter body 402, as desired.

In some instances, the jet orifices 418 may be oriented at an angle relative to the longitudinal axis of the supply tube wall 412. For example, the proximally projecting jet orifices 418a-c may be oriented at an oblique (e.g., acute) angle relative to the longitudinal axis of the supply tube wall 412 and/or oriented at an angle greater than zero degrees and less than ninety degrees relative to the longitudinal axis of the supply tube wall 412. It is contemplated that a distally projecting jet orifice 418d may be oriented at an oblique (e.g., obtuse) angle relative to the longitudinal axis of the supply tube wall 412 and/or oriented at an angle greater than 90 degrees and less than 180 degrees relative to the longitudinal axis of the supply tube wall 412. In other instances, the jet orifices 418 may be oriented perpendicular to the longitudinal axis of the supply tube wall 412 (e.g., at an angle of about 90 degrees relative to the longitudinal axis of the supply tube wall 412). The angle may or may not be the same for all the jet orifices 418.

In at least some instances, the jet orifices 418 may be understood as being arranged in series. In other words, the jet orifices 418 may be arranged such that adjacent jet orifices 418 are spaced longitudinally apart at various locations along the longitudinal axis of the supply tube wall 412. For example, the jet orifices 418 may be uniformly or non-uniformly spaced along of a length of the supply tube wall 412. This may position the jet orifices 418 at axially spaced apart locations within the catheter body 402 and along the length thereof. For example, the jet orifices 418 may be spaced along an entire length of the supply tube wall 412 and correspondingly along an entire length of the catheter body 402, or portions thereof, as desired. In some examples, the jet orifices 418 may be spaced at intervals in the range of every 5 inches (12.7 centimeters (cm)) to every 15 inches (38.1 cm), or in the range of every 6 inches (15.2 cm) to every 12 inches (30.5 cm) along a length of the supply tube wall 412. In other instances, the spacing between the jet orifices 418 may be less than every 5 inches (12.7 cm) or greater than every 15 inches (38.1 cm). Accordingly, motive fluid leaves via the jet orifices 418 forming a jetted motive fluid 420a-d (collectively, 420). In some instances, the jetted motive fluid 420 may reach speeds of 17,150 centimeters/second or greater (e.g., half the speed of sound, or greater). This jetted motive fluid 420 enters an entrainment material where the shear layer between the two causes turbulence, mixing, and transfer of momentum. Entrainment material may enter the distal opening 408 and then may be urged proximally by momentum transfer. As the mixture of jetted motive fluid 420 and entrainment material migrates proximally, the material may sequentially approach a number of jet orifices 418. Upon interaction with the jetted motive fluid 420 from each individual jet orifice 418, the momentum in the entrainment material mixture may increase, and the thrombogenic material may more readily flow proximally through the catheter body 402 for removal. The increase in momentum may allow for the catheter body 402 to be used without a second or outflow orifice (e.g., positioned proximally of the distal opening 408). Alternatively, some of the entrapped thrombogenic material may exit the catheter body 402 through a second orifice (not shown), e.g., in a sidewall of the catheter body 402, positioned proximal to the distal opening 408, recirculate to the distal opening 408 (e.g., one or more times), and then move proximally through the lumen 406 of the catheter body 402.

It is further contemplated that the distally oriented motive jetted fluid 420d may be partially to fully entrained by the force generated by the proximally oriented motive jetted fluid 420a-c. When the clot/thrombus reaches the distally oriented motive jetted fluid 420d, the shear stress may masticate the clot/thrombus. It is contemplated that when the distal opening 408 of the catheter body 402 is sealed with a clot/thrombus, the force generated by the proximally oriented motive jetted fluid 420a-c may be transferred to the surface of the clot/thrombus in a proximal direction. As a result, the distally oriented motive jetted fluid 420d may no longer be entrained and may transfer force in the distal direction to the surface of the clot/thrombus. Thus, when the distal opening 408 is clogged or plugged, an extreme shear mechanism of action is created where the distal and proximal force vectors combine together to focus all of the shear stress to the surface of the clot/thrombus to masticate the clot/thrombus and unplug the distal opening 408. It is contemplated that the shear stress on the clot/thrombus may be much larger in magnitude when the distally oriented motive jetted fluid 420d is at a smaller angle (e.g., closer to 180 degrees relative to the longitudinal axis of the supply tube wall 412 than to orthogonal to the longitudinal axis of the supply tube wall 412).

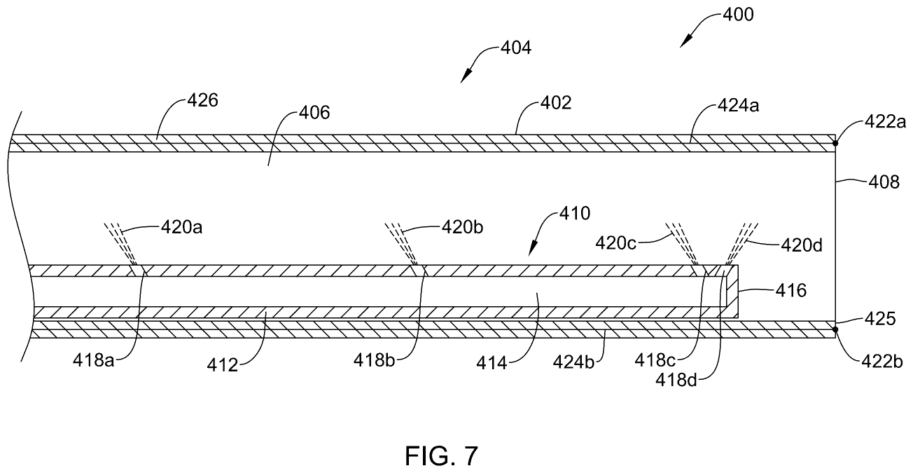

FIG. 7 illustrates the thrombectomy catheter 400 of FIG. 6 further including a first electrode 422a and a second electrode 422b positioned along the distal end region of the catheter body 402. FIG. 7 further illustrates the first electrode 422a positioned on the distal facing surface 425 of the wall 426 of the catheter body 402 and a second electrode 422b positioned on the distal facing surface 425 of the wall 426 of the catheter body 402. It can be appreciated that the first electrode 422a may be radially, axially and/or circumferentially spaced (e.g., offset, etc.) from the second electrode 422b at any position (e.g., longitudinal position) along the distal facing surface 425 of the catheter body 402.

FIG. 7 further illustrates that the first electrode 422a may be coupled to the processor 332 (shown in FIG. 5A) via a first wire 424a and the that the second electrode 422b may be coupled to the processor 332 via a second wire 424b. It can be appreciated that the first electrode 422a and the second electrode 422b may be similar in form and function as the first electrode 322a and the second electrode 322b described with respect to the thrombectomy catheter 300. For example, the processor 332 may sense an impedance measurement of the bodily substance (e.g., clot, plaque, thrombus, blood, etc.) between the first electrode 422a and the second electrode 422b and send a signal (via the transmitter 334) to the console 330 to turn the pump 56 on/off depending on the sensed impedance measurement.

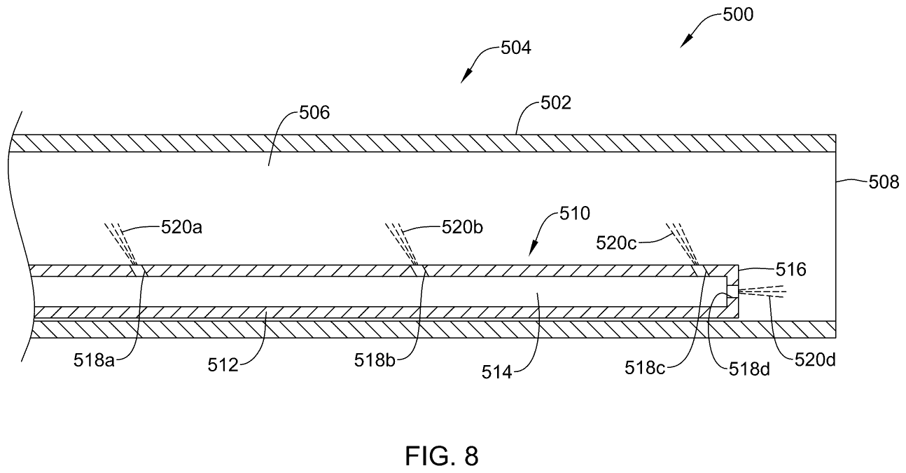

FIG. 8 is a cross-sectional view of a distal end region 504 of another illustrative thrombectomy catheter 500. The thrombectomy catheter 500 may be one illustrative example of the thrombectomy catheter 58 described above. The thrombectomy catheter 500 may include a tubular member or catheter body 502 extending from a proximal end region (not explicitly shown) configured to remain outside the body to a distal end region 504. The catheter body 502 may be one illustrative example of, or be in fluid communication with, the effluent return tube 66 of the thrombectomy catheter 58 described above. A lumen 506 may extend from the proximal end region to the distal end region 504 of the catheter body 502. The catheter body 502 may terminate at a distally facing distal opening 508 at the distal end of the catheter body 502. In some instances, the distal opening 508 may be in a plane that extends generally orthogonal to a longitudinal axis of the catheter body 502. In other instances, the distal opening 508 may be in a plane that extends generally oblique to a longitudinal axis of the catheter body 502. Generally, the distal opening 508 may be an entrainment inflow orifice. While not explicitly shown, the catheter body 502 may include one or more markers (e.g., radiopaque marker bands) disposed along the catheter body 502. Further, while not explicitly shown, in some embodiments, the catheter body 502 may include one or more openings extending through a sidewall thereof, if desired.

The thrombectomy catheter 500 may further include a high-pressure fluid supply tube 510. The high-pressure fluid supply tube 510 may be one illustrative example of, or be in fluid communication with, the high-pressure fluid supply tube 66 of the thrombectomy catheter 58 described above. The high-pressure fluid supply tube 510 may be disposed within and extend through the lumen 506 of the catheter body 502. The high-pressure fluid supply tube 510 may include a supply tube wall 512 defining a lumen or fluid pathway 514 extending therethrough. In at least some instances, the high-pressure fluid supply tube 510 may have a partially closed distal end 516. The high-pressure fluid supply tube 510 may extend along a length of the catheter body 502 with the distal end 516 located within the lumen 506 of the catheter body 502 proximal to the distal opening 508 at the distal end of the catheter body 502. A proximal end of the high-pressure fluid supply tube 510 may be in fluid communication with the pump 56 described herein, to provide high-pressure fluid to the fluid pathway 514 of the high-pressure fluid supply tube 510.

A plurality of jet orifices 518a-d (collectively, 518) may be defined along the supply tube wall 512. For example, the supply tube wall 512 may include two, three, four, five, six, or more jet orifices 518. The jet orifices 518 may be spaced along the supply tube wall 512 at any desired intervals. For example, each of the jet orifices 518 may be equidistantly spaced from adjacent jet orifices 518 along the length of the supply tube wall 512. In other instances, the jet orifices 518 may be arranged such that the spacing between adjacent jet orifices 518 near the distal end of the supply tube wall 512 is closer than the spacing between adjacent jet orifices 518 near the proximal end of the supply tube wall 512. For instance, the spacing between the orifices 518 may gradually increase as you move proximally along the length of the shaft, or the spacing may increase in a step-wise configuration. In some instances, some or all of the jet orifices 518 may be axially aligned along the supply tube wall 512. In other instances, one or more of the jet orifices 518 may be circumferentially offset from one another about the supply tube wall 512. A number of patterns are contemplated including a helical pattern, a pattern where no two jet orifices 518 are disposed at the same axial location, a regular pattern including two or more jet orifices 518 disposed at the same axial location, an irregular pattern (where some of the jet orifices 518 may or may not be disposed at the same axial location), etc. The jet orifices 518 may be formed using a suitable method such as electron discharge machining, etching, cutting (e.g., including laser cutting), or the like. In some instances, one or more of the jet orifices 518 may have a substantially round shape. In other instances, one or more of the jet orifices 518 may have a substantially non-round shape (e.g., oval, polygonal, irregular, etc.). In some instances, the jet orifices 518 may be beveled or otherwise include a beveled surface. It is contemplated that a size and/or a shape of the jet orifices 518 may be varied to vary the velocity of the fluid exiting the jet orifices. For example, decreasing the size of the jet orifices 518 may increase the velocity of the fluid exiting the jet orifices 518. In some embodiments, the size of the jet orifices 518 may be varied based on the pressure capacity of the thrombectomy system, the number of jet orifices, the dimensions of the high-pressure fluid supply tube 510 (e.g., length, wall thickness, inner diameter, etc.), and/or combinations thereof. In some examples, the jet orifices 518 may have a cross-sectional dimension in the range of about 0.0018 inches (45.72 micrometers) to about 0.0022 inches (55.88 micrometers). However, the jet orifices 518 can have a cross-sectional dimension of less than 0.0018 inches (45.72 micrometers) or greater than 0.0022 inches (55.88 micrometers), as desired.

Infusion of motive fluid through the lumen 514 of the supply tube wall 512 may result in fluid being jetted through the jet orifices 518 and the generation of a proximally directed aspiration force. At least some of the jet orifices 518a-c may extend through a circumferential sidewall of the supply tube wall 512. It is contemplated that at least some of the jet orifices 518a-c may be angled in a proximal direction or otherwise designed to infuse fluid (e.g., a motive fluid, a liquid, a gas or air, steam, a fluid with particles disposed therein, or the like) through the jet orifices 518a-c and into the lumen 506 of the catheter body 502 in a generally proximal direction as depicted by lines 520a-c representing motive jetted fluid projecting generally proximally from the jet orifices 518a-c. For example, each of the jet orifices 518a-c may be arranged at an acute angle to the longitudinal axis of the supply tube wall 512 such that the jet orifices 518a-c angle in a proximal direction. In some embodiments, one or more of the jet orifices 518d may be designed to infuse fluid (e.g., a motive fluid, a liquid, a gas or air, steam, a fluid with particles disposed therein, or the like) through the jet orifice(s) 518d and into the lumen 506 of the catheter body 502 in a generally distal direction as depicted by lines 520d representing motive jetted fluid projecting generally distally from the jet orifice 518d. For example, the jet orifice 518d may be arranged in line with the longitudinal axis of the supply tube wall 512 such that the jet orifice 518d expels fluid distally in a direction generally parallel to the longitudinal axis of the supply tube wall 512.

As further described herein, the supply tube wall 512 may include one or more, or a plurality of proximally oriented or directed jet orifices 518a, 518b, 518c (i.e., jet orifices configured to direct fluid infused through the lumen 514 of the supply tube wall 512 in a proximal direction) and the supply tube wall 512 may include one or more, or a plurality of distally oriented or directed jet orifices 518d (i.e., jet orifices configured to direct fluid infused through the lumen 514 of the supply tube wall 512 in a distal direction).

It is contemplated that the jet orifice 518d may be formed though the distal end 516 of the supply tube wall 512. The distally projecting jet orifice 518d may have diameter that is less than an inner diameter of the lumen 514 of the supply tube wall 512. In other examples, a diameter of the distally projecting jet orifice 518d may be approximately the same as an inner diameter of the lumen 514 of the supply tube wall 512. In yet other examples, the circumferential sidewalls of the supply tube wall 512 may be beveled at the distal end 516 such that a diameter of the distally projecting jet orifice 518d may be greater than a diameter of the lumen 514 of the supply tube wall 512. It is contemplated that the distal end 516 of the supply tube wall 512 may be proximal to the distal opening 508 of the catheter body 502 to fully entrain the distally projecting jet orifice 518d.

The distally projecting jet orifice 518d may be the distalmost jet orifice, with the proximally projecting jet orifices 518a-c positioned proximal of the distally projecting jet orifice 518d. While the supply tube wall 512 is illustrated as including only a single distally projecting jet orifice 518d, the supply tube wall 512 may include more than one distally projecting jet orifice, as desired. In some cases, more than one distally projecting jet orifices 518d may be formed in the distal end 516 of the supply tube wall 512. Alternatively, or additionally, one or more additional distally projecting jet orifices 518d may be formed in a circumferential sidewall of the supply tube wall 512. When more than one distally projecting jet orifice 518d is provided, one or more distally projecting jet orifices may extend through a circumferential sidewall of the supply tube wall 512 at differing axial and/or circumferential locations from one another or similar axial and/or circumferential locations as one another, as desired. The distally projecting jet orifice(s) 518d may break up particles as they are drawn into the lumen 506 of the catheter body 502 while the proximally projecting jet orifices 518a-c may move particles proximally along the catheter body 502.

The performance of the thrombectomy catheter 500 and the high-pressure fluid supply tube 510 may be directly related to the velocity of the motive jetted fluid 520 exiting the jet orifices 518 and the shear-induced turbulent flux created by the jetted motive fluid 520. For example, the more powerful the jetted motive fluid 520, the higher the aspiration rates may be. It is further contemplated that the performance of the jet-powered aspiration catheter 500 may be directly related to the speed at which the clot can be entrained into the catheter 500, macerated, and removed from the body. Any clogging that occurs within the catheter body 502 may reduce or completely stop the removal of the clot. The addition of the distally projecting jet orifice 518d may macerate any clot that enters the distal opening 508 of the catheter body 502 thus helping prevent clogging. For example, at the point of impingement of the distally oriented motive jetted fluid 520d the motive jetted fluid 520d may deflect distally creating flow out the tip of the distal opening 508 of the catheter body 502, effectively macerating any clot that enters the distal opening 508 of the catheter body 502 and eliminating or reducing risk of the distal opening 508 of the catheter body 502 becoming blocked or clogged. It is contemplated that the properties (size, shape, angle, number, spacing, etc.) of the jet orifices 518 may be varied to obtain a fluid velocity that creates an optimum de-clogging effect without hindering the proximal flow of a clot within the lumen 506 of the catheter body 502 or the clot evacuation rate.

The distally projecting jet orifice 518d may be proximally spaced a distance from the distal opening 508 of the catheter body 502. It is contemplated that the longitudinal location of the distally projecting jet orifice 518d on the supply tube wall 512 and relative to the distal opening 508 of the catheter body 502 may be varied based on a size of the aperture of the distally projecting jet orifice 518d, the velocity of the fluid within the lumen 514 of the supply tube wall 512, the angle of the distally projecting jet orifice 518d, or combinations thereof, etc. to ensure the distally oriented motive jetted fluid 520d does not impinge the vessel wall.

In some instances, the jet orifices 518 may be oriented at an angle relative to the longitudinal axis of the supply tube wall 512. For example, the proximally projecting jet orifices 518a-c may be oriented at an oblique (e.g., acute) angle relative to the longitudinal axis of the supply tube wall 512 and/or oriented at an angle greater than zero degrees and less than ninety degrees relative to the longitudinal axis of the supply tube wall 512. It is contemplated that a distally projecting jet orifice 518d may be oriented along or parallel to the longitudinal axis of the supply tube wall 512. In other instances, the jet orifices 518 may be oriented perpendicular to the longitudinal axis of the supply tube wall 512 (e.g., at an angle of about 90 degrees relative to the longitudinal axis of the supply tube wall 512). The angle may or may not be the same for all the jet orifices 518. It is contemplated that an angle of the jet orifices 518 and thus the motive jetted fluid 520 may be varied by to adjust the velocity of the fluid exiting the jet orifices 518.

In at least some instances, the jet orifices 518 may be understood as being arranged in series. In other words, the jet orifices 518 may be arranged such that adjacent jet orifices 518 are spaced longitudinally apart at various locations along the longitudinal axis of the supply tube wall 512. For example, the jet orifices 518 may be uniformly or non-uniformly spaced along of a length of the supply tube wall 512. This may position the jet orifices 518 at axially spaced apart locations within the catheter body 502 and along the length thereof. For example, the jet orifices 518 may be spaced along an entire length of the supply tube wall 512 and correspondingly along an entire length of the catheter body 502, or portions thereof, as desired. In some examples, the jet orifices 518 may be spaced at intervals in the range of every 5 inches (12.7 centimeters (cm)) to every 15 inches (38.1 cm), or in the range of every 6 inches (15.2 cm) to every 12 inches (30.5 cm) along a length of the supply tube wall 512. In other instances, the spacing between the jet orifices 518 may be less than every 5 inches (12.7 cm) or greater than every 15 inches (38.1 cm). Accordingly, motive fluid leaves via the jet orifices 518 forming a jetted motive fluid 520a-d (collectively, 520). In some instances, the jetted motive fluid 520 may reach speeds of 17,150 centimeters/second or greater (e.g., half the speed of sound, or greater). This jetted motive fluid 520 enters an entrainment material where the shear layer between the two causes turbulence, mixing, and transfer of momentum. Entrainment material may enter the distal opening 508 and then may be urged proximally by momentum transfer. As the mixture of jetted motive fluid 520 and entrainment material migrates proximally, the material may sequentially approach a number of jet orifices 518. Upon interaction with the jetted motive fluid 520 from each individual jet orifice 518, the momentum in the entrainment material mixture may increase, and the thrombogenic material may more readily flow proximally through the catheter body 502 for removal. The increase in momentum may allow for the catheter body 502 to be used without a second or outflow orifice (e.g., positioned proximally of the distal opening 508). Alternatively, some of the entrapped thrombogenic material may exit the catheter body 502 through a second orifice (not shown), e.g., in a sidewall of the catheter body 502, positioned proximal to the distal opening 508, recirculate to the distal opening 508 (e.g., one or more times), and then move proximally through the lumen 506 of the catheter body 502.

It is further contemplated that the distally oriented motive jetted fluid 520d may be partially to fully entrained by the force generated by the proximally oriented motive jetted fluid 520a-c. When the clot/thrombus reaches the distally oriented motive jetted fluid 520d, the shear stress may masticate the clot/thrombus. It is contemplated that when the distal opening 508 of the catheter body 502 is sealed with a clot/thrombus, the force generated by the proximally oriented motive jetted fluid 520a-c may be transferred to the surface of the clot/thrombus in a proximal direction. As a result, the distally oriented motive jetted fluid 520d may no longer be entrained and may transfer force in the distal direction to the surface of the clot/thrombus. Thus, when the distal opening 508 is clogged or plugged, an extreme shear mechanism of action is created where the distal and proximal force vectors combine together to focus all of the shear stress to the surface of the clot/thrombus to masticate the clot/thrombus and unplug the distal opening 508.

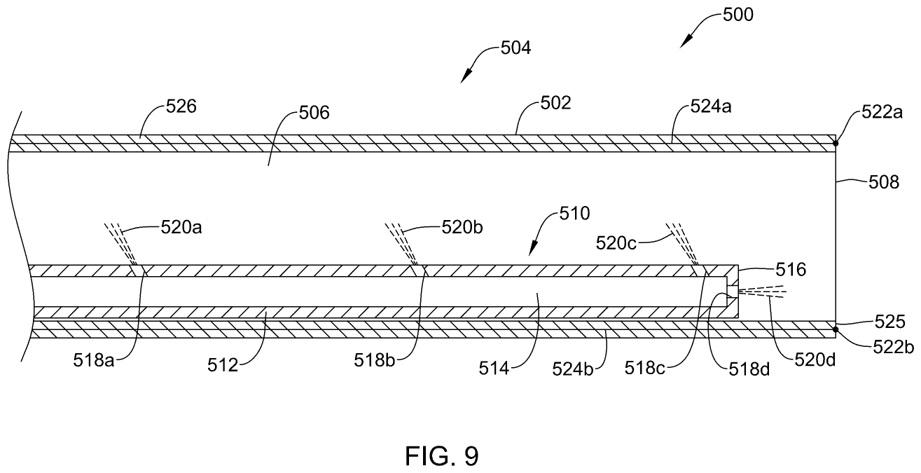

FIG. 9 illustrates the thrombectomy catheter 500 of FIG. 8 further including a first electrode 522a and a second electrode 522b positioned along the distal end region 504 of the catheter body 502. FIG. 9 further illustrates the first electrode 522a positioned on the distal facing surface 525 of the wall 526 of the catheter body 502 and a second electrode 522b positioned on the distal facing surface 525 of the wall 526 of the catheter body 502. It can be appreciated that the first electrode 522a may be radially, axially and/or circumferentially spaced (e.g., offset, etc.) from the second electrode 522b at any position (e.g., longitudinal position) along the distal facing surface 525 of the catheter body 502.

FIG. 9 further illustrates that the first electrode 522a may be coupled to the processor 332 (shown in FIG. 5A) via a first wire 524a and the that the second electrode 522b may be coupled to the processor 332 via a second wire 524b. It can be appreciated that the first electrode 522a and the second electrode 522b may be similar in form and function as the first electrode 322a and the second electrode 322b described with respect to the thrombectomy catheter 300. For example, the processor 332 may sense an impedance measurement of the bodily substance (e.g., clot, plaque, thrombus, blood, etc.) between the first electrode 522a and the second electrode 522b and send a signal (via the transmitter 334) to the console 330 to turn the pump 56 on/off depending on the sensed impedance measurement.

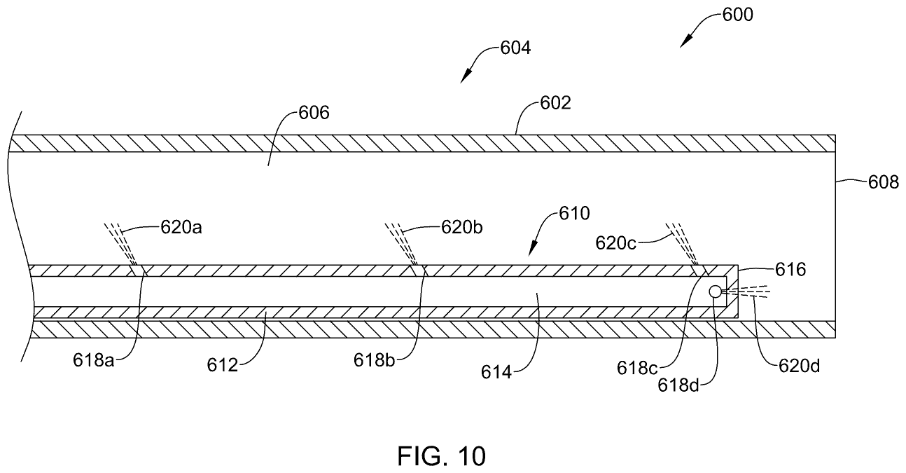

FIG. 10 is a cross-sectional view of a distal end region 604 of another illustrative thrombectomy catheter 600. The thrombectomy catheter 600 may be one illustrative example of the thrombectomy catheter 58 described above. The thrombectomy catheter 600 may include a tubular member or catheter body 602 extending from a proximal end region (not explicitly shown) configured to remain outside the body to a distal end region 604. The catheter body 602 may be one illustrative example of, or be in fluid communication with, the effluent return tube 66 of the thrombectomy catheter 58 described above. A lumen 606 may extend from the proximal end region to the distal end region 604 of the catheter body 602. The catheter body 602 may terminate at a distally facing distal opening 608 at the distal end of the catheter body 602. In some instances, the distal opening 608 may be in a plane that extends generally orthogonal to a longitudinal axis of the catheter body 602. In other instances, the distal opening 608 may be in a plane that extends generally oblique to a longitudinal axis of the catheter body 602. Generally, the distal opening 608 may be an entrainment inflow orifice. While not explicitly shown, the catheter body 602 may include one or more markers (e.g., radiopaque marker bands) disposed along the catheter body 602. Further, while not explicitly shown, in some embodiments, the catheter body 602 may include one or more openings extending through a sidewall thereof, if desired.

The thrombectomy catheter 600 may further include a high-pressure fluid supply tube 610. The high-pressure fluid supply tube 610 may be one illustrative example of, or be in fluid communication with, the high-pressure fluid supply tube 66 of the thrombectomy catheter 58 described above. The high-pressure fluid supply tube 610 may be disposed within and extend through the lumen 606 of the catheter body 602. The high-pressure fluid supply tube 610 may include a supply tube wall 612 defining a lumen or fluid pathway 614 extending therethrough. In at least some instances, the high-pressure fluid supply tube 610 may have a closed distal end 616. Because of this, fluid may be able to pass through the fluid pathway 614 but does not exit the distal end. The high-pressure fluid supply tube 610 may extend along a length of the catheter body 602 with the distal end 616 located within the lumen 606 of the catheter body 602 proximal to the distal opening 608 at the distal end of the catheter body 602. A proximal end of the high-pressure fluid supply tube 610 may be in fluid communication with the pump 56 described herein, to provide high-pressure fluid to the fluid pathway 614 of the high-pressure fluid supply tube 610.

A plurality of jet orifices 618a-d (collectively, 618) may be defined along the supply tube wall 612. For example, the supply tube wall 612 may include two, three, four, five, six, or more jet orifices 618. The jet orifices 618 may be spaced along the supply tube wall 612 at any desired intervals. For example, each of the jet orifices 618 may be equidistantly spaced from adjacent jet orifices 618 along the length of the supply tube wall 612. In other instances, the jet orifices 618 may be arranged such that the spacing between adjacent jet orifices 618 near the distal end of the supply tube wall 612 is closer than the spacing between adjacent jet orifices 618 near the proximal end of the supply tube wall 612. For instance, the spacing between the orifices 618 may gradually increase as you move proximally along the length of the shaft, or the spacing may increase in a step-wise configuration. In some instances, some or all of the jet orifices 618 may be axially aligned along the supply tube wall 612. In other instances, one or more of the jet orifices 618 may be circumferentially offset from one another about the supply tube wall 612. A number of patterns are contemplated including a helical pattern, a pattern where no two jet orifices 618 are disposed at the same axial location, a regular pattern including two or more jet orifices 618 disposed at the same axial location, an irregular pattern (where some of the jet orifices 618 may or may not be disposed at the same axial location), etc. The jet orifices 618 may be formed using a suitable method such as electron discharge machining, etching, cutting (e.g., including laser cutting), or the like. In some instances, one or more of the jet orifices 618 may have a substantially round shape. In other instances, one or more of the jet orifices 618 may have a substantially non-round shape (e.g., oval, polygonal, irregular, etc.). In some instances, the jet orifices 618 may be beveled or otherwise include a beveled surface. It is contemplated that a size and/or a shape of the jet orifices 618 may be varied to vary the velocity of the fluid exiting the jet orifices. For example, decreasing the size of the jet orifices 618 may increase the velocity of the fluid exiting the jet orifices 618. In some embodiments, the size of the jet orifices 618 may be varied based on the pressure capacity of the thrombectomy system, the number of jet orifices, the dimensions of the high-pressure fluid supply tube 610 (e.g., length, wall thickness, inner diameter, etc.), and/or combinations thereof. In some examples, the jet orifices 618 may have a cross-sectional dimension in the range of about 0.0018 inches (45.72 micrometers) to about 0.0022 inches (55.88 micrometers). However, the jet orifices 608 can have a cross-sectional dimension of less than 0.0018 inches (45.72 micrometers) or greater than 0.0022 inches (55.88 micrometers), as desired.

Infusion of motive fluid through the lumen 614 of the supply tube wall 612 may result in fluid being jetted through the jet orifices 618 and the generation of a proximally directed aspiration force. At least some of the jet orifices 618a-c may extend through a circumferential sidewall of the supply tube wall 612. It is contemplated that at least some of the jet orifices 618a-c may be angled in a proximal direction or otherwise designed to infuse fluid (e.g., a motive fluid, a liquid, a gas or air, steam, a fluid with particles disposed therein, or the like) through the jet orifices 618a-c and into the lumen 606 of the catheter body 602 in a generally proximal direction as depicted by lines 620a-c representing motive jetted fluid projecting generally proximally from the jet orifices 618a-c. For example, each of the jet orifices 618a-c may be arranged at an acute angle to the longitudinal axis of the supply tube wall 612 such that the jet orifices 618a-c angle in a proximal direction. In some embodiments, one or more of the jet orifices 618d may be designed to infuse fluid (e.g., a motive fluid, a liquid, a gas or air, steam, a fluid with particles disposed therein, or the like) through the jet orifice(s) 618d and into the lumen 606 of the catheter body 602 in a generally distal direction as depicted by lines 620d representing motive jetted fluid projecting generally distally from the jet orifice 618d. For example, the jet orifice 618d may be arranged at an oblique angle to the longitudinal axis of the supply tube wall 612 such that the jet orifice 618d angles in a distal direction.

As further described herein, the supply tube wall 612 may include one or more, or a plurality of proximally oriented or directed jet orifices 618a, 618b, 618c (i.e., jet orifices configured to direct fluid infused through the lumen 614 of the supply tube wall 612 in a proximal direction) and the supply tube wall 612 may include one or more, or a plurality of distally oriented or directed jet orifices 618d (i.e., jet orifices configured to direct fluid infused through the lumen 614 of the supply tube wall 612 in a distal direction).

In some embodiments, the distally projecting jet orifice 618d may be circumferentially offset from the proximally projecting jet orifices 618a-d. In the illustrated embodiment, the distally projecting jet orifice 618d may be spaced in the range of about 45° to about 135° or approximately 90° about the circumference of the supply tube wall 612 from the proximally projecting jet orifices 618a-c. However, other circumferential spacing intervals may be used, as desired. For example, the distally projecting jet orifice 618d may be spaced in the range of about 10° to about 350°, about 45° to about 135°, or about 60° to about 120° from the proximally projecting jet orifices 618a-d. However, it is contemplated that the distally projecting jet orifice 618d may not be positioned adjacent the catheter body 602. It is contemplated that positioning the distally projecting jet orifice 618d circumferentially offset from the proximal projecting jet orifices 618a-c may create a spiral effect with motive jetted fluid 620 thus increasing mastication of the clots or debris.

The distally projecting jet orifice 618d may be the distalmost jet orifice, with the proximally projecting jet orifices 618a-c positioned proximal of the distally projecting jet orifice 618d. However, this is not required. In some embodiments, the distally projecting jet orifice 618d may be positioned proximal to at least one proximally projecting jet orifice 618a-c. While the supply tube wall 612 is illustrated as including only a single distally projecting jet orifice 618d, the supply tube wall 612 may include more than one distally projecting jet orifice, as desired. When more than one distally projecting jet orifice 618d is provided, the distally projecting jet orifices may be positioned at differing axial and/or circumferential locations from one another or similar axial and/or circumferential locations as one another, as desired. The distally projecting jet orifice(s) 618d may break up particles as they are drawn into the lumen 606 of the catheter body 602 while the proximally projecting jet orifices 618a-c may move particles proximally along the catheter body 602.

The performance of the thrombectomy catheter 600 and the high-pressure fluid supply tube 610 may be directly related to the velocity of the motive jetted fluid 620 exiting the jet orifices 618 and the shear-induced turbulent flux created by the jetted motive fluid 620. For example, the more powerful the jetted motive fluid 620, the higher the aspiration rates may be. It is further contemplated that the performance of the jet-powered aspiration catheter 600 may be directly related to the speed at which the clot can be entrained into the catheter 600, macerated, and removed from the body. Any clogging that occurs within the catheter body 602 may reduce or completely stop the removal of the clot. The addition of the distally projecting jet orifice 618d may macerate any clot that enters the distal opening 608 of the catheter body 602 thus helping prevent clogging. For example, at the point of impingement of the distally oriented motive jetted fluid 620d the motive jetted fluid 620d may deflect distally creating flow out the tip of the distal opening 608 of the catheter body 602, effectively macerating any clot that enters the distal opening 608 of the catheter body 602, and eliminating or reducing risk of the distal opening 608 of the catheter body 602 becoming blocked or clogged. It is contemplated that the properties (size, shape, angle, number, spacing, etc.) of the jet orifices 618 may be varied to obtain a fluid velocity that creates an optimum de-clogging effect without hindering the proximal flow of a clot within the lumen 606 of the catheter body 602 or the clot evacuation rate.

The distally projecting jet orifice 618d may be proximally spaced a distance from the distal opening 608 of the catheter body 602. It is contemplated that the longitudinal location of the distally projecting jet orifice 618d on the supply tube wall 612 and relative to the distal opening 608 of the catheter body 602 may be varied based on a size of the aperture of the distally projecting jet orifice 618d, the velocity of the fluid within the lumen 614 of the supply tube wall 612, the angle of the distally projecting jet orifice 618d, or combinations thereof, etc. to ensure the distally oriented motive jetted fluid 620d impinges the inner surface of the catheter body 602. In one illustrative example, the distally projecting jet orifice 618d may be positioned such that the distally oriented motive jetted fluid 620d impinges an inner surface of the catheter body 602 such that the distally oriented motive jetted fluid 620d does not damage the vessel. For example, the distally projecting jet orifice 618d may be positioned such that the distally oriented motive jetted fluid 620d impinges an inner surface of the catheter body 602 in the range of about 0.070 inches (1.778 millimeters) to about 0.090 inches (2.286 millimeters) proximal to the distal end of the catheter body 602. This is just one example. The impingement location of the motive jetted fluid 620d of the distally projecting jet orifice 418d may be less than 0.070 inches (1.778 millimeters) or more than 0.090 inches (2.286 millimeters) proximal to the distal end of the catheter body 602, as desired.

In some instances, the jet orifices 618 may be oriented at an angle relative to the longitudinal axis of the supply tube wall 612. For example, the sidewalls of the proximally projecting jet orifices 618a-c may be oriented at an oblique (e.g., acute) angle relative to the longitudinal axis of the supply tube wall 612 and/or oriented at an angle greater than zero degrees and less than ninety degrees relative to the longitudinal axis of the supply tube wall 612. It is contemplated that the sidewalls of a distally projecting jet orifice 618d may be oriented at an oblique (e.g., obtuse) angle relative to the longitudinal axis of the supply tube wall 612 and/or oriented at an angle greater than 90 degrees and less than 180 degrees relative to the longitudinal axis of the supply tube wall 612. In other instances, the jet orifices 618 may be oriented perpendicular to the longitudinal axis of the supply tube wall 612 (e.g., at an angle of about 90 degrees relative to the longitudinal axis of the supply tube wall 612). The angle may or may not be the same for all the jet orifices 618. It is contemplated that an angle of the jet orifices 618 and thus the motive jetted fluid 620 may be varied by to adjust the velocity of the fluid exiting the jet orifices 618.

In at least some instances, at least some of the jet orifices 618 may be understood as being arranged in series. In other words, the jet orifices 618 may be arranged such that adjacent jet orifices 618 are spaced longitudinally apart at various locations along the longitudinal axis of the supply tube wall 612. For example, the jet orifices 618 may be uniformly or non-uniformly spaced along of a length of the supply tube wall 612. This may position the jet orifices 618 at axially spaced apart locations within the catheter body 602 and along the length thereof. For example, the jet orifices 618 may be spaced along an entire length of the supply tube wall 612 and correspondingly along an entire length of the catheter body 602, or portions thereof, as desired. In some examples, the jet orifices 618 may be spaced at intervals in the range of every 5 inches (12.7 centimeters (cm)) to every 15 inches (38.1 cm), or in the range of every 6 inches (15.2 cm) to every 12 inches (30.5 cm) along a length of the supply tube wall 612. In other instances, the spacing between the jet orifices 618 may be less than every 5 inches (12.7 cm) or greater than every 15 inches (38.1 cm). Accordingly, motive fluid leaves via the jet orifices 618 forming a jetted motive fluid 620a-d (collectively, 620). In some instances, the jetted motive fluid 620 may reach speeds of 17,150 centimeters/second or greater (e.g., half the speed of sound, or greater). This jetted motive fluid 620 enters an entrainment material where the shear layer between the two causes turbulence, mixing, and transfer of momentum. Entrainment material may enter the distal opening 608 and then may be urged proximally by momentum transfer. As the mixture of jetted motive fluid 620 and entrainment material migrates proximally, the material may sequentially approach a number of jet orifices 618. Upon interaction with the jetted motive fluid 620 from each individual jet orifice 618, the momentum in the entrainment material mixture may increase, and the thrombogenic material may more readily flow proximally through the catheter body 602 for removal. The increase in momentum may allow for the catheter body 602 to be used without a second or outflow orifice (e.g., positioned proximally of the distal opening 608). Alternatively, some of the entrapped thrombogenic material may exit the catheter body 602 through a second orifice (not shown), e.g., in a sidewall of the catheter body 602, positioned proximal to the distal opening 608, recirculate to the distal opening 608 (e.g., one or more times), and then move proximally through the lumen 606 of the catheter body 602.

It is further contemplated that the distally oriented motive jetted fluid 620d may be partially to fully entrained by the force generated by the proximally oriented motive jetted fluid 620a-c. When the clot/thrombus reaches the distally oriented motive jetted fluid 620d, the shear stress may masticate the clot/thrombus. It is contemplated that when the distal opening 608 of the catheter body 602 is sealed with a clot/thrombus, the force 20) generated by the proximally oriented motive jetted fluid 620a-c may be transferred to the surface of the clot/thrombus in a proximal direction. As a result, the distally oriented motive jetted fluid 620d may no longer be entrained and may transfer force in the distal direction to the surface of the clot/thrombus. Thus, when the distal opening 608 is clogged or plugged, an extreme shear mechanism of action is created where the distal and proximal force vectors combine together to focus all of the shear stress to the surface of the clot/thrombus to masticate the clot/thrombus and unplug the distal opening 608.

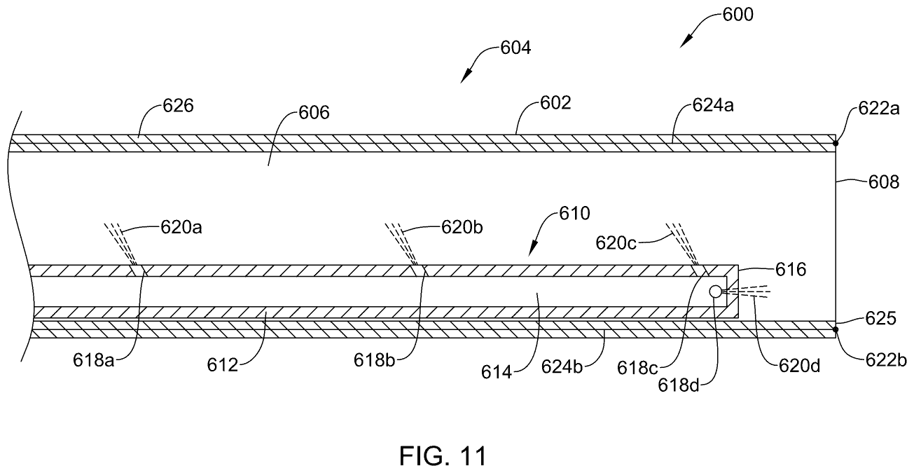

FIG. 11 illustrates the thrombectomy catheter 600 of FIG. 10 further including a first electrode 622a and a second electrode 622b positioned along the distal end region 604 of the catheter body 602. FIG. 11 further illustrates the first electrode 622a positioned on the distal facing surface 625 of the wall 626 of the catheter body 602 and the second electrode 622b positioned on the distal facing surface 625 of the wall 626 of the catheter body 602. It can be appreciated that the first electrode 622a may be radially, axially and/or circumferentially spaced (e.g., offset, etc.) from the second electrode 622b at any position (e.g., longitudinal position) along the distal facing surface 625 of the catheter body 602.

FIG. 11 further illustrates that the first electrode 622a may be coupled to the processor 332 (shown in FIG. 5A) via a first wire 624a and the that the second electrode 622b may be coupled to the processor 332 via a second wire 624b. It can be appreciated that the first electrode 622a and the second electrode 622b may be similar in form and function as the first electrode 322a and the second electrode 322b described with respect to the thrombectomy catheter 300. For example, the processor 332 may sense an impedance measurement of the bodily substance (e.g., clot, plaque, thrombus, blood, etc.) between the first electrode 622a and the second electrode 622b and send a signal (via the transmitter 334) to the console 330 to turn the pump 56 on/off depending on the sensed impedance measurement.

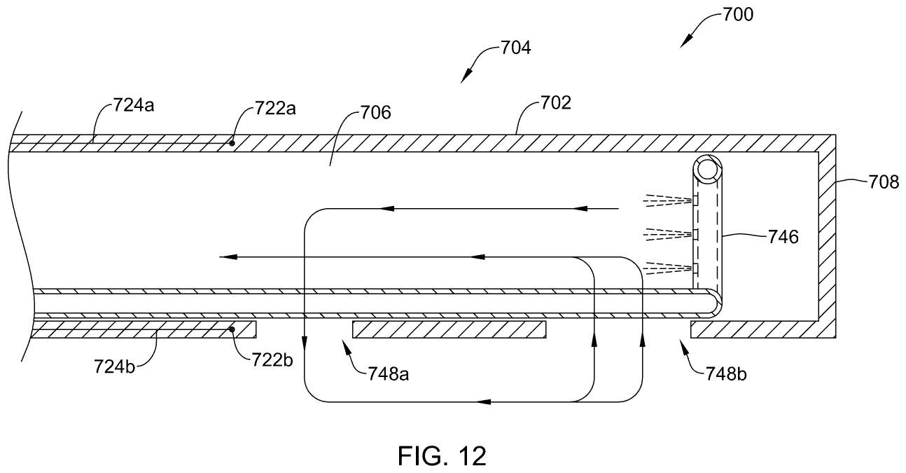

FIG. 12 is a cross-sectional view of another example thrombectomy catheter 700. The thrombectomy catheter 700 may be one illustrative example of the thrombectomy catheter 58 described above. The thrombectomy catheter 700 may include a tubular catheter shaft 702, a fluid jet emanatory 746 and one or more opening(s) 748 along the side wall of the catheter shaft. The catheter body 702 may have a closed distal end 708. An example thrombectomy system can be found in U.S. Pat. No. 8,162,877, the entirety of which is incorporated by reference. Other suitable thrombectomy systems may include The AngioJet Infusion Systems, available from Boston Scientific.

FIG. 12 illustrates the thrombectomy catheter 700 of FIG. 12 further includes a first electrode 722a and a second electrode 722b positioned proximal to an outflow opening 748a of the catheter body 702. In some examples, the first electrode 722a and a second electrode 722b may be positioned between the outflow opening 748a and the inflow opening 748b. FIG. 12 further illustrates that the first electrode 722a may be coupled to the processor 332 (shown in FIG. 5) via a first wire 724a and the that the second electrode 722b may be coupled to the processor 332 via a second wire 724b.

While FIG. 12 illustrates that the first electrode 722a and the second electrode 722b positioned within the wall of the catheter body 702, it can be appreciated that the first electrode 722a, the second electrode 722b or both the first electrode 722a and the second electrode 722b may be positioned substantially flush with an inner surface of the catheter body 702. Further, it can be appreciated that the first electrode 722a, the second electrode 722b or both the first electrode 722a and the second electrode 722b may be positioned substantially flush with an outer surface of the catheter body 702. Further, it can be appreciated that the first wire 724a, the second wire 724b or both the first wire 724a and the second wire 724b may be positioned substantially flush with an inner surface of the catheter body 702. Further, it can be appreciated that the first wire 724a, the second wire 724b or both the first wire 724a and the second wire 724b may be positioned substantially flush with an outer surface of the catheter body 702.

It can be appreciated that the first electrode 722a and the second electrode 722b may be similar in form and function as the first electrode 322a and the second electrode 322b described with respect to the thrombectomy catheter 300. For example, the processor 332 may sense an impedance measurement of the bodily substance (e.g., clot, plaque, thrombus, blood, etc.) between the first electrode 722a and the second electrode 722b and send a signal (via the transmitter 334) to the console 330 to turn the pump 56 on/off depending on the sensed impedance measurement.

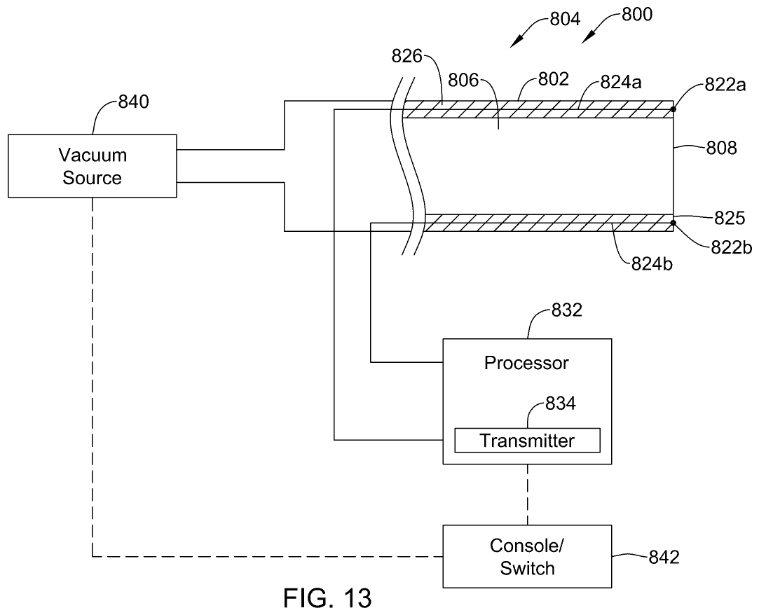

FIG. 13 illustrates a cross-sectional view of the distal end region of an example aspiration catheter 800. The aspiration catheter 800 may include a tubular member or catheter body 802 extending from a proximal end region (not explicitly shown) configured to remain outside the body to a distal end region 804. A proximal end of the aspiration catheter 800 may be in fluid communication with a vacuum source 840 (e.g., the pump 56 described herein), to withdraw the fluid, tissue, or other bodily substance (e.g., clot, plaque, thrombus, blood, etc.) through the lumen 806 of the catheter 800. A lumen 806 may extend from the proximal end region to the distal end region 804 of the catheter body 802. The catheter body 802 may terminate at a distally facing distal opening 808 at the distal end of the catheter body 802. In some instances, the distal opening 808 may be in a plane that extends generally orthogonal to a longitudinal axis of the catheter body 802. In other instances, the distal opening 808 may be in a plane that extends generally oblique to a longitudinal axis of the catheter body 802.

FIG. 13 further illustrates that the aspiration catheter 800 may include a first electrode 822a positioned within the wall 826 of the catheter body 820 and a second electrode 822b positioned within the wall 826 of the catheter body 820. It can be appreciated that the first electrode 822a may be radially, axially and/or circumferentially spaced (e.g., offset, etc.) from the second electrode 822b at any position (e.g., longitudinal position) along the catheter body 802.

FIG. 13 further illustrates that the first electrode 822a and the second electrode 822b maybe positioned along a distal facing surface 825 of the catheter body 802 (the position of the first electrode 822a and the second electrode 822b along the distal facing surface 825 is further illustrated in FIG. 13). It can be appreciated that while FIG. 13 illustrates that the first electrode 822a and/or the second electrode 822b may be positioned along the distal facing surface 825 of the catheter body 802, the first electrode 822a and/or the second electrode 822b may be positioned axially along any portion of the distal end region 804 of the catheter body 802. For example, the first electrode 822a and/or the second electrode 822b may be positioned at any location proximally of the distal end of the catheter body 802 (e.g., the first electrode 822a and/or the second electrode 822b may be positioned within the wall 826 of the proximal body 802 at any position proximal of the distal facing surface 825 of the catheter body 802).

Further, FIG. 13 illustrates that the first electrode 822a may be attached to a first 20) wire 824a. The first wire 824a may be positioned within the wall 826 of the catheter body 802. Additionally, FIG. 13 illustrates that the second electrode 822b may be attached to a second wire 824b. The second wire 824b may also be positioned within the wall 826 of the catheter body 802.

While FIG. 13 illustrates that the first electrode 822a and the second electrode 822b positioned within the wall 826 of the catheter body 802, it can be appreciated that the first electrode 822a, the second electrode 822b or both the first electrode 822a and the second electrode 822b may be positioned substantially flush with an inner surface of the catheter body 802. Further, it can be appreciated that the first electrode 822a, the second electrode 822b or both the first electrode 822a and the second electrode 822b may be positioned substantially flush with an outer surface of the catheter body 802. Further, it can be appreciated that the first wire 824a, the second wire 824b or both the first wire 824a and the second wire 824b may be positioned substantially flush with an inner surface of the catheter body 802. Further, it can be appreciated that the first wire 824a, the second wire 824b or both the first wire 824a and the second wire 824b may be positioned substantially flush with an outer surface of the catheter body 802.

FIG. 13 further illustrates that the first wire 824a and the second wire 824b may extend way from the first electrode 822a, 822b, respectively, through the wall 826 of the catheter body and eventually be coupled (e.g., be attached) to a processor 832. FIG. 13 illustrates that the processor 832 may be coupled to a switch and/or console 842. As used herein, the switch 842 may define any device used to open and close an electric circuit, whereby opening/closing the electrical circuit may start or stop a pump configured to draw fluid into the lumen 806. As described herein with respect to other systems (e.g., the system 10), the switch 842 may be positioned within a console. In other examples, the switch 842 may be a component separate from a console. It can be appreciated that, in some examples, the processor 832 may be a distinct component spaced away from the switch 842. The processor 832 may be configured to communicate wirelessly (e.g., via Bluetooth communication, etc.) with the switch 842. In other examples, the processor 832 may communicate with the switch 842 via a hardwire connection. It can be further appreciated that, in other examples, the processor 832 may be integrated into a component with the switch 842. In other words, the processor 832 and the switch 842 may be located within a component of the system 10, including a console or the drive unit 12 shown in FIG. 1.

FIG. 13 further illustrates that the processor 832 may include a signal transmitter 834. Additionally, the signal transmitter 834 may be configured to transport a signal (or create a pathway for a signal) to travel from the processor 832 to the switch 842 or a component coupled to the switch 842.

FIG. 13 further illustrates that the first electrode 822a may be coupled to the processor 832 (shown in FIG. 13) via a first wire 824a and the that the second electrode 822b may be coupled to the processor 832 via a second wire 824b. It can be appreciated that the first electrode 822a and the second electrode 822b may be similar in form and function as the first electrode 322a and the second electrode 322b described with respect to the thrombectomy catheter 300. For example, the processor 832 may sense an impedance measurement of the bodily substance (e.g., clot, plaque, thrombus, blood, etc.) between the first electrode 822a and the second electrode 822b and send a signal (via the transmitter 834) to the switch 842 to turn the pump 56 on/off depending on the sensed impedance measurement or a comparison of multiple sensed impedance measurements.

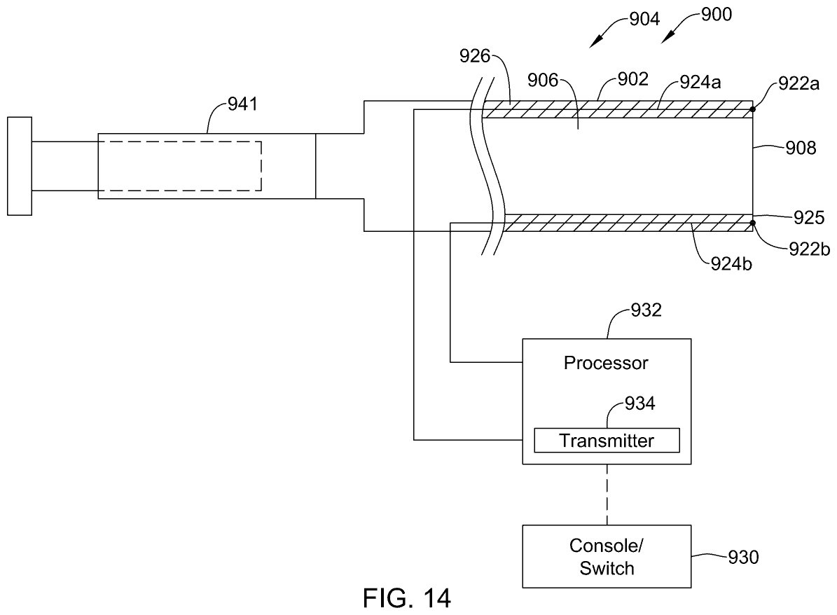

FIG. 14 illustrates a cross-sectional view of the distal end region of another example aspiration catheter 900. The aspiration catheter 900 may include a tubular member or catheter body 902 extending from a proximal end region (not explicitly shown) configured to remain outside the body to a distal end region 904. A proximal end of the aspiration catheter 900 may be in fluid communication with a syringe 941, to withdraw the fluid, tissue, or other bodily substance (e.g., clot, plaque, thrombus, blood, etc.) through the lumen 906 of the catheter 900. A lumen 906 may extend from the proximal end region to the distal end region 904 of the catheter body 902. The catheter body 902 may terminate at a distally facing distal opening 908 at the distal end of the catheter body 902. In some instances, the distal opening 908 may be in a plane that extends generally orthogonal to a longitudinal axis of the catheter body 902. In other instances, the distal opening 908 may be in a plane that extends generally oblique to a longitudinal axis of the catheter body 902.

FIG. 14 further illustrates that the aspiration catheter 900 may include a first electrode 922a positioned within the wall 926 of the catheter body 920 and a second electrode 922b positioned within the wall 926 of the catheter body 920. It can be appreciated that the first electrode 922a may be radially, axially and/or circumferentially spaced (e.g., offset, etc.) from the second electrode 922b at any position (e.g., longitudinal position) along the catheter body 902. FIG. 14 further illustrates that the first electrode 922a and the second electrode 922b maybe positioned along a distal facing surface 925 of the catheter body 902 (the position of the first electrode 922a and the second electrode 922b along the distal facing surface 925 is further illustrated in FIG. 14). It can be appreciated that while FIG. 14 illustrates that the first electrode 922a and/or the second electrode 922b may be positioned along the distal facing surface 925 of the catheter body 902, the first electrode 922a and/or the second electrode 922b may be positioned axially along any portion of the distal end region 904 of the catheter body 902. For example, the first electrode 922a and/or the second electrode 922b may be positioned at any location proximally of the distal end of the catheter body 902 (e.g., the first electrode 922a and/or the second electrode 922b may be positioned within the wall 926 of the proximal body 902 at any position proximal of the distal facing surface 925 of the catheter body 902).

Further, FIG. 14 illustrates that the first electrode 922a may be attached to a first wire 924a. The first wire 924a may be positioned within the wall 926 of the catheter body 902. Additionally, FIG. 14 illustrates that the second electrode 922b may be attached to a second wire 924b. The second wire 924b may also be positioned within the wall 926 of the catheter body 902.

While FIG. 14 illustrates that the first electrode 922a and the second electrode 922b positioned within the wall 926 of the catheter body 902, it can be appreciated that the first electrode 822a, the second electrode 922b or both the first electrode 922a and the second electrode 922b may be positioned substantially flush with an inner surface of the catheter body 902. Further, it can be appreciated that the first electrode 922a, the second electrode 922b or both the first electrode 922a and the second electrode 922b may be positioned substantially flush with an outer surface of the catheter body 902. Further, it can be appreciated that the first wire 924a, the second wire 924b or both the first wire 924a and the second wire 924b may be positioned substantially flush with an inner surface of the catheter body 902. Further, it can be appreciated that the first wire 924a, the second wire 924b or both the first wire 924a and the second wire 924b may be positioned substantially flush with an outer surface of the catheter body 902.

FIG. 14 further illustrates that the first wire 924a and the second wire 924b may extend way from the first electrode 922a, 922b, respectively, through the wall 926 of the catheter body and eventually be coupled (e.g., be attached) to a processor 932. FIG. 14 illustrates that the processor 932 may be coupled to a switch and/or console 942. As used herein, the switch 942 may define any device used to open and close an electric circuit, whereby opening/closing the electrical circuit may start or stop a pump configured to draw fluid into the lumen 906. As described herein with respect to other systems (e.g., the system 10), the switch 942 may be positioned within a console. In other examples, the switch 942 may be a component separate from a console. It can be appreciated that, in some examples, the processor 932 may be a distinct component spaced away from the switch 942. The processor 932 may be configured to communicate wirelessly (e.g., via Bluetooth communication, etc.) with the switch 942. In other examples, the processor 932 may communicate with the switch 942 via a hardwire connection. It can be further appreciated that, in other examples, the processor 832 may be integrated into a component with the switch 942. In other words, the processor 932 and the switch 942 may be located within a component of the system 10, including a console or the drive unit 12 shown in FIG. 1.

FIG. 14 further illustrates that the processor 932 may include a signal transmitter 934. Additionally, the signal transmitter 934 may be configured to transport a signal (or create a pathway for a signal) to travel from the processor 932 to the switch 942 or a component coupled to the switch 942.

FIG. 14 further illustrates that the first electrode 922a may be coupled to the processor 932 (shown in FIG. 14) via a first wire 924a and the that the second electrode 922b may be coupled to the processor 932 via a second wire 924b. It can be appreciated that the first electrode 922a and the second electrode 922b may be similar in form and function as the first electrode 322a and the second electrode 322b described with respect to the thrombectomy catheter 300. For example, the processor 932 may sense an impedance measurement of the bodily substance (e.g., clot, plaque, thrombus, blood, etc.) between the first electrode 922a and the second electrode 922b and send a signal (via the transmitter 934) to the switch 942 to turn the pump 56 on/off depending on the sensed impedance measurement or a comparison of multiple sensed impedance measurements.

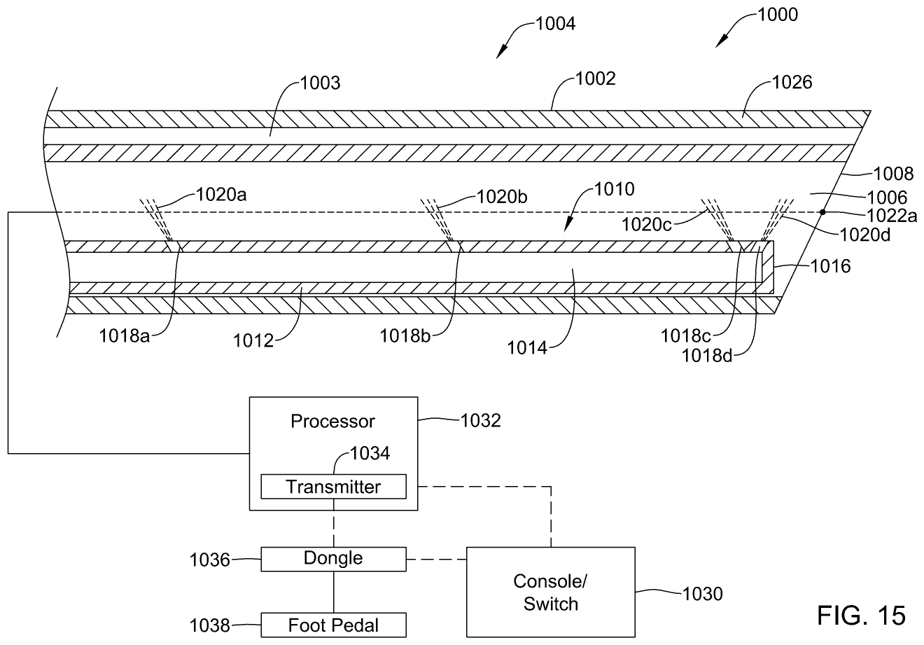

FIG. 15 illustrates a cross-sectional view of the distal end region of an example thrombectomy catheter 1000. The thrombectomy catheter 1000 may be similar in form and function to any of thrombectomy catheters disclosed herein. For example, the thrombectomy catheter 1000 may include a tubular member or catheter body 1002 extending from a proximal end region (not explicitly shown) configured to remain outside the body to a distal end region 1004. The catheter body 1002 may be one illustrative example of, or be in fluid communication with, the effluent return tube 66 of the thrombectomy catheter 58 described above. A lumen 1006 may extend from the proximal end region to the distal end region 1004 of the catheter body 1002. Further, a guidewire lumen 1003 may extend from the proximal end region to the distal end region 1004 of the catheter body 1002. While not explicitly shown, the catheter body 1002 may include one or more markers (e.g., radiopaque marker bands) disposed along the catheter body 1002.

Additionally, the catheter body 1002 may terminate at a distally facing distal opening 1008 at the distal end of the catheter body 1002. In some instances, distal end of the catheter body 1002 may include a taper. For example, the end of the catheter body 1002 may be in a plane that extends at an angle relative to the longitudinal axis of the catheter body 1002 (e.g., generally oblique to a longitudinal axis of the catheter body 1002). However, in other examples, the end of the catheter body 1002 may be generally orthogonal to a longitudinal axis of the catheter body 1002.

The thrombectomy catheter 1000 may further include a high-pressure fluid supply tube 1010. The high-pressure fluid supply tube 310 may be one illustrative example of, or be in fluid communication with, the high-pressure fluid supply tube 66 of the thrombectomy catheter 58 described above. The high-pressure fluid supply tube 1010 may be disposed within and extend through the lumen 1006 of the catheter body 1002. The high-pressure fluid supply tube 1010 may include a supply tube wall 1012 defining a lumen or fluid pathway 1014 extending therethrough. In at least some instances, the high-pressure fluid supply tube 1010 may have a closed distal end 1016. Because of this, fluid may be able to pass distally through the fluid pathway 1014 but does not exit the distal end. The high-pressure fluid supply tube 1010 may extend along a length of the catheter body 1002 with the distal end 1016 located within the lumen 1006 of the catheter body 1002 proximal to the distal opening 1008 at the distal end of the catheter body 1002. A proximal end of the high-pressure fluid supply tube 1010 may be in fluid communication with the pump 56 described herein, to provide high-pressure fluid to the fluid pathway 1014 of the high-pressure fluid supply tube 1010.

FIG. 15 illustrates at the thrombectomy catheter 1000 may include one or more jet orifices 1018 which may be defined along the supply tube wall 1012. While only one jet orifice is depicted in FIG. 15, it can be appreciated that the supply tube wall 1012 may include one, two, three, four, five, six, or more jet orifices 1018 (similar in form and function to the jet orifices 1018a-d disclosed above with respect to the thrombectomy catheter 400). Like that described herein with respective the catheter 400, infusion of motive fluid through the lumen 1014 of the supply tube wall 1012 may result in fluid being jetted through the jet orifices (e.g., the one or more jet orifices 1018) and the generation of a proximally directed aspiration force through the catheter body 1012. Further, entrainment material may enter the distal opening 1008 and then may be urged proximally by momentum transfer. As the mixture of jetted motive fluid 1020 and entrainment material moves proximally, the material may sequentially approach a number of jet orifices (e.g., the one or more jet orifices 1018) positioned along the supply tube wall 1012. Upon interaction with the jetted motive fluid 1020 from each individual jet orifice 1018, the momentum in the entrainment material mixture may increase, and the thrombogenic material may more readily flow proximally through the catheter body 1002 for removal.

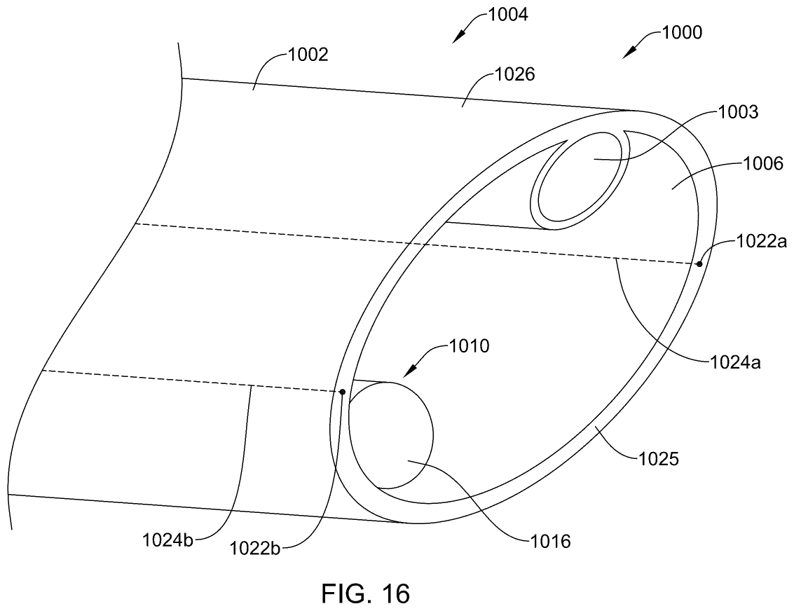

FIG. 15 further illustrates that the thrombectomy catheter 1000 may include a first electrode 1022a positioned within the wall 1026 of the catheter body 1002 and a second electrode 1022b (shown in FIG. 16) positioned within the wall 1026 of the catheter body 1002. It can be appreciated that the first electrode 1022a may be radially, axially and/or circumferentially spaced (e.g., offset, etc.) from the second electrode 1022b at any position (e.g., longitudinal position) along the catheter body 1002. FIG. 15 further illustrates that the first electrode 1022a and the second electrode 1022b maybe positioned along a distal facing surface 1025 of the catheter body 1002 (the position of the first electrode 1022a and the second electrode 1022b along the distal facing surface 1025 is further illustrated in FIG. 16). It can be appreciated that while FIG. 15 illustrates that the first electrode 1022a and/or the second electrode 1022b may be positioned along the distal facing surface 1025 of the catheter body 1002, the first electrode 1022a and/or the second electrode 1022b may be positioned axially along any portion of the distal end region 1004 of the catheter body 1002. For example, the first electrode 1022a and/or the second electrode 1022b may be positioned at any location proximally of the distal end of the catheter body 1002 (e.g., the first electrode 1022a and/or the second electrode 1022b may be positioned within the wall 1026 of the proximal body 1002 at any position proximal of the distal facing surface 1025 of the catheter body 1002).

Further, FIG. 15 illustrates that the first electrode 1022a may be attached to a first wire 1024a. The first wire 1024a may be positioned within the wall 1026 of the catheter body 1002. Additionally, FIG. 16 illustrates that the second electrode 1022b may be attached to a second wire 1024b. The second wire 1024b may also be positioned within the wall 1026 of the catheter body 1002.

While FIG. 15 illustrates that the first electrode 1022a and the second electrode 1022b positioned within the wall 1026 of the catheter body 1002, it can be appreciated that the first electrode 1022a, the second electrode 1022b or both the first electrode 1022a and the second electrode 1022b may be positioned substantially flush with an inner surface of the catheter body 1002. Further, it can be appreciated that the first electrode 1022a, the second electrode 1022b or both the first electrode 1022a and the second electrode 1022b may be positioned substantially flush with an outer surface of the catheter body 1002. Further, it can be appreciated that the first wire 1024a, the second wire 1024b or both the first wire 1024a and the second wire 1024b may be positioned substantially flush with an inner surface of the catheter body 1002. Further, it can be appreciated that the first wire 1024a, the second wire 1024b or both the first wire 1024a and the second wire 1024b may be positioned substantially flush with an outer surface of the catheter body 1002.

FIGS. 15-16 further illustrates that the first wire 1024a and the second wire 1024b may extend way from the first electrode 1022a, 1022b, respectively, through the wall 1026 of the catheter body and eventually be coupled (e.g., be attached) to a processor 1032. FIG. 15 illustrates that the processor 1032 may be coupled to a switch and/or console 1030. It can be appreciated that, in some examples, the processor 1032 may be a distinct component spaced away from the console 1030. The processor 1032 may be configured to communicate wirelessly (e.g., via Bluetooth communication, etc.) with console 1030. In other examples, the processor 1032 may communicate with the console 1030 via a hardwire connection. It can be further appreciated that, in other examples, the processor 1032 may be integrated into the console 1030. In other words, the processor 1032 may be located within the control console or drive unit 12 shown in FIG. 1.

FIG. 15 further illustrates that the processor 1032 may include a signal transmitter 1034. Additionally, the signal transmitter 1034 may be configured to transport a signal (or create a pathway for a signal) to travel from the processor 1032 to the console 1030 or a component (e.g., switch, etc.) coupled to the console 1030. For example, FIG. 15 illustrates that the transmitter 1034 may be configured to send a signal to a dongle 1036 which is coupled to the console 1030. As will be described in greater detail herein, the signal May trigger the dongle 1036 to open or close an electrical circuit which starts/stops the pump 56 within the console 1030. FIG. 15 further illustrates that a foot pedal 1038 may also be coupled to the dongle 1038, whereby the foot pedal 1038 is configured to send a signal to the dongle 1036. The signal sent from the foot pedal 1038 to the dongle 1036 may cause the dongle 1036 to open or close an electrical circuit which starts/stops the pump 56 within the console 1030.

Additionally, the first electrode 1022a and the second electrode 1022b may operate in a bipolar configuration to sense (e.g., measure, detect, etc.) parameter (e.g., impedance, pressure, force, etc.) of a bodily substance (e.g., tissue, plaque, thrombus, blood, etc.) positioned between the first electrode 1022a and the second electrode 1022b. For example, it can be appreciated that thrombectomy system 1000 may be configured to send an electrical signal from the processor 1032 to the first electrode 1024a via the first wire 1024a, whereby the electrical signal passes from the first electrode 1024a, through the bodily substance present at the distal end region of the catheter body 1002 (e.g., through the bodily substance positioned between the first electrode 1022a and the second electrode 1022b), whereby the electrical signal is then received by the second electrode 1022b. It can further be appreciated that the electrical signal may then be passed from the second electrode 1022b back to the processor 1032 via the second wire 1024b. Both the first electrode 1022a and the second electrode 1022b electrodes contribute to the measured impedance because the electrical current passes from one electrode to the other through the small volume of bodily substance between them.

Additionally, the processor 1032 may be programmed to compare the signal sent from the processor 1032 to first electrode 1022a to the signal received from the second electrode 1022b. The resultant value may provide a measurement of the impedance (e.g., opposition to electrical flow) of the bodily substance positioned between the first electrode 1022a and the second electrode 1022b. Further, the processor 1032 may be configured to compare the measured impedance to approximate, preset (e.g., preprogrammed) ranges for the impedance of blood versus the impedance of a clot (e.g., plaque, thrombus, etc.). The preset (e.g., preprogrammed) ranges for the impedance of blood versus the impedance of a clot (e.g., plaque, thrombus, etc.) may be stored in the memory or processing componentry 1032 of the system 1000. In other examples, the ranges for the impedance of blood versus the impedance of a clot (e.g., plaque, thrombus, etc.) may be input into the system 1000 by a clinician via a touchpad on the console 1030.

It can be further appreciated that the system 1000 may be configured such that when the measured impedance is within a range which indicates the tip of the catheter 1000 is adjacent to a clot (e.g., plaque, thrombus, etc.), the transmitter 1034 may send a signal (e.g., a wireless signal) to the dongle 1036, whereby the dongle 1036 may close an electrical circuit which than starts the pump 56. As discussed herein, starting the pump 56 may cause infusion of motive fluid through the lumen 1014 of the supply tube wall 1012, resulting in fluid being jetted through the jet orifices (e.g., the one or more jet orifices 1018) and the generation of a proximally directed aspiration force through the catheter body 1012 to remove the clot (e.g., plaque, thrombus, etc.).

In other examples, the catheter 1000 may include one or more blood-sensing electrodes and/or sensors positioned along the catheter 1000 whereby the blood-sensing electrodes or sensors may be adjacent to blood when the distal tip of the catheter 1000 is adjacent to a clot (e.g., plaque, thrombus, etc.). The blood-sensing electrodes and/or sensors may be similar in form and function to the first electrode 322a and/or the second electrode 322b described herein. For example, like the first electrode 1022a and the second electrode 1022b, the blood-sensing electrodes may be able to measure the impedance of blood when the distal tip of the catheter 1000 is adjacent to a clot (e.g., plaque, thrombus, etc.). Further, the system 1000 may be configured such that the impedance of material adjacent to the distal tip of the catheter 1000 may be compared to the impedance of blood measured by the blood-sensing electrodes. Accordingly, the processor 1032 may be configured to measure and compute the ratio of the impedance of material adjacent to the distal tip of the catheter 1000 compared to the impedance of blood measured by the blood-sensing electrodes. Further, this resultant ratio value may indicate that the tip of the catheter 1000 is adjacent to a clot (e.g., plaque, thrombus, etc.). For example, the processor 1032 may be configured to compare this calculated ratio to approximate, preset (e.g., preprogrammed) ranges for the ratio of the impedance of material adjacent to the distal tip of the catheter 1000 compared to the impedance of blood measured by the blood-sensing electrodes. The preset (e.g., preprogrammed) ranges for the calculated ratio may be stored in the memory or processing componentry 1032 of the system 1000. In other examples, the calculated ration may be input into the system 1000 by a clinician via a touchpad on the console 1030.

It can be further appreciated that the system 1000 may be configured such that when the calculated ration is within a range which indicates the tip of the catheter 1000 is adjacent to a clot (e.g., plaque, thrombus, etc.), the transmitter 1034 may send a signal (e.g., a wireless signal) to the dongle 1036, whereby the dongle 1036 may close an electrical circuit which than starts the pump 56. As discussed herein, starting the pump 56 may cause infusion of motive fluid through the lumen 1014 of the supply tube wall 1012, resulting in fluid being jetted through the jet orifices (e.g., the one or more jet orifices 1018) and the generation of a proximally directed aspiration force through the catheter body 1012 to remove the clot (e.g., plaque, thrombus, etc.).

It can be appreciated that after the thrombectomy catheter 1000 has removed the clot, plaque, thrombus, etc., blood may fill the distal tip of the catheter 1000 between the first electrode 1022a and the second electrode 1022a may be blood, and therefore, the impedance measurement sensed by processor 1032 may change. Accordingly, when the measured impedance is within a range which indicates the tip of the catheter 1000 is adjacent to a blood (which has a different impedance than clot, plaque, thrombus), the transmitter 1034 may send a signal (e.g., a wireless signal) to the dongle 1036, whereby the dongle 1036 may open an electrical circuit which than stops the pump 56. As discussed herein, stopping the pump 56 may stop the infusion of motive fluid through the lumen 1014 of the supply tube wall 1012, preventing the continued removal of blood from the patient.

Additionally, it can be appreciated that, in some examples, the signal transmitter 1034 may be configured to transport a signal (or create a pathway for a signal) to travel from the processor 1032 directly to the console 1030 ((bypassing the dongle 1036, for example). For example, FIG. 15 illustrates that the transmitter 1034 may be configured to send a signal directly to a receiver positioned in the console 1030. As will be described in greater detail herein, the signal may trigger the receiver (or another component within the console 1030 that receives the signal) to start/stop the pump 56 within the console 1030. Further, in some examples, the drive unit 12 of the thrombectomy system 10 may plug directly into the console 1030, whereby the drive unit 12 may transmit a signal which triggers a receiver (or other component within the console 1030 that receives the signal) to start/stop the pump 56 within the console 1030. In yet other examples, the console 1030 may include a Wi-Fi adaptor, whereby the Wi-Fi adaptor is configured to receive a signal from another component of the thrombectomy system 1000 (e.g., the drive unit 12, the processor 1032, the transmitter 1034, etc.), and whereby the signal received by the Wi-Fi adaptor may trigger the starting or stopping of the pump 56.

It can be further appreciated that when the system 1000 may be defined as being configured to “automatically” sense the presence of clot, plaque, thrombus or the like when the system 1000 is configured such that the processor continuously senses the impedance of the bodily material positioned adjacent the distal tip of the catheter 1000 (e.g., senses the impedance of bodily material between the first electrode 1022a and the second electrode 1022b), and subsequently causes the transmitter to send signals to the console 1030 and/or dongle 1036 to turn the pump 56 on/off. In some instances, the console 1030 may include a switch, button, actuator, etc. which permits a clinician to turn the “automatic” impedance sensing operation of the system 1000 on or off. For example, the console 1036 may include an on/off switch which must be turned on by the clinician in order for the system 1000 to operate in the “automatic impedance sensing” configuration, whereby by the pump 56 is automatically turned on and off depending on the impedance measurements sensed by the processor 1032.

The processor 1032 may be configured to continually measure the impedance of bodily material positioned between the first electrode 1022a and the second electrode 1022b. Accordingly, the processor 1032 may be configured to receive and process impedance signals such that it may start and stop the pump 56 with a single pump stroke.

As discussed herein, the system 1000 may be designed to include a foot pedal 1038 coupled to the dongle 1036. It can be appreciated that the foot pedal 1038 may permit a clinician to activate the pump 56, and hence, the flow fluid through the lumen 1014. In some instances, the foot pedal 1038 may be utilized to override a signal coming from the processor 1032 (via the transmitter 1034). For example, in some instances when the clinician is operating the system 1000 in the “automatic” operating configuration as described herein, the clinician may want to activate the pump 56 despite the processor 1032 not having sensed an impedance which would automatically active that pump 56. In other words, depressing the foot pedal may permit the clinician to manually activate the pump 56 even though the system 1000 is in the “automatic” operating configuration as described herein.

FIG. 16 is a perspective view of the distal end region 1004 of the example thrombectomy catheter 1000 shown in FIG. 15. As discussed herein, the thrombectomy catheter 1000 may include a tubular member or catheter body 1002 extending from a proximal end region (not explicitly shown) configured to remain outside the body to a distal end region 1004. FIG. 16 further illustrates the guidewire lumen 1003 and the high-pressure fluid supply tube 1010 discussed herein. FIG. 16 further illustrates the tapered distal end of the catheter body 1002 as described herein.

As discussed herein, FIG. 16 further illustrates the first electrode 1022a positioned on the distal facing surface 1025 of the wall 1026 of the catheter body 1002 and a second electrode 1022b positioned on the distal facing surface 1025 of the wall 1026 of the catheter body 1002. It can be appreciated that the first electrode 1022a may be radially, axially and/or circumferentially spaced (e.g., offset, etc.) from the second electrode 1022b at any position (e.g., longitudinal position) along the distal facing surface 1025 of the catheter body 1002.

Using the analogy of a 12-hour clock face, FIG. 16 illustrates that the first electrode 1022a and/or the second electrode 1022b may be positioned along the distal facing surface 1025 at approximately a 9 o'clock and 3 o'clock position. However, this is not intended to be limiting. Rather, for any of the example aspiration and/or thrombectomy systems described herein, the first electrode 1022a and/or the second electrode 1022b may be positioned at any location along the distal face 1025 or within the wall 1026 of the catheter body 1026.

The materials that can be used for the various components of the thrombectomy catheter, pump/catheter assembly, and/or other devices disclosed herein may include those commonly associated with medical devices. For simplicity purposes, the following discussion refers to the pump/catheter assembly and its related components. However, this is not intended to limit the devices and methods described herein, as the discussion may be applied to other similar devices, tubular members and/or components of tubular members or devices disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An aspiration catheter, comprising:
a catheter body having a proximal end region, a distal end region and a lumen extending therein; and
an impedance sensor disposed along the distal end region of the catheter body, the impedance sensor configured to sense the impedance difference between blood and thrombus.

2. The aspiration catheter of claim 1, wherein the impedance sensor includes a first electrode and a second electrode.

3. The aspiration catheter of claim 2, wherein the first electrode is radially spaced away from the second electrode along the catheter body.

4. The aspiration catheter of claim 2, wherein the first electrode, the second electrode or both the first electrode and the second electrode are positioned substantially flush with an inner surface of the catheter body.

5. The aspiration catheter of claim 2, wherein the first electrode, the second electrode or both the first electrode and the second electrode are positioned substantially flush with an outer surface of the catheter body.

6. The aspiration catheter of claim 1, further comprising a fluid supply tube including at least one proximally projecting jet orifice for expelling at least one proximally oriented fluid jet from the fluid supply tube within the catheter lumen in a generally proximal direction.

7. The aspiration catheter of claim 6, wherein the fluid supply tube includes at least one distally projecting jet orifice for expelling at least one distally oriented fluid jet from the fluid supply tube within the catheter lumen in a generally distal direction.

8. The aspiration catheter of claim 7, wherein the at least one distally projecting jet orifice is distal to the at least one proximally projecting jet orifice.

9. The aspiration catheter of claim 8, wherein the at least one distally projecting jet orifice extends through a sidewall of the fluid supply tube.

10. The aspiration catheter of claim 7, wherein the catheter body further includes an entrainment inflow orifice positioned along the distal end region of the catheter body.

11. A thrombectomy system, comprising:
a processor coupled to a pump and a thrombectomy catheter, wherein the thrombectomy catheter includes:
a catheter body having a proximal end region, a distal end region and a lumen extending therein;
a fluid supply tube extending within the lumen of the catheter body and coupled to the pump; and
an impedance sensor disposed along the distal end region of the catheter body;
wherein the processor is configured to sense a change in impedance of a first bodily substance adjacent to the impedance sensor.

12. The thrombectomy system of claim 11, wherein the impedance sensor includes a first electrode and a second electrode.

13. The thrombectomy system of claim 11, wherein the processor is configured to sense and compare the impedance of the first bodily substance with a second bodily substance.

14. The thrombectomy system of claim 13, wherein the first bodily substance is blood.

15. The thrombectomy system of claim 14, wherein the second bodily substance is thrombus.

16. The thrombectomy system of claim 13, wherein the processor is configured to send a signal to the pump based on the comparison of the impedance of the first bodily substance with the impedance of the second bodily substance.

17. The thrombectomy system of claim 16, wherein the pump is configured to inject fluid through the fluid supply tube based on the signal received from the processor.

18. The thrombectomy system of claim 17, wherein the processor is wirelessly coupled to the pump.

19. A thrombectomy system, comprising:
a processor coupled to a fluid pump and a thrombectomy catheter, wherein the thrombectomy catheter includes:
a catheter body having a proximal end region, a distal end region and a lumen extending therein;
a fluid supply tube extending within the lumen of the catheter body and coupled to the pump, wherein the fluid supply tube includes at least one proximally projecting jet orifice for expelling at least one proximally oriented fluid jet from the fluid supply tube within the catheter lumen in a generally proximal direction, and wherein the fluid supply tube includes at least one distally projecting jet orifice for expelling at least one distally oriented fluid jet from the fluid supply tube within the catheter lumen in a generally distal direction; and
an impedance sensor in communication with the processor, the impedance sensor disposed along the distal end region of the catheter body;
wherein the processor is configured to sense the impedance of a bodily substance positioned adjacent to the impedance sensor.

20. The thrombectomy system of claim 19, the processor is configured to sense the impedance difference between blood and thrombus.
